(19) United States
(12) Patent Application Publication
(10) Pub. No.: US 2011/0098254 A1
(43) Pub. Date: Apr. 28, 2011

(54) COMPOSITIONS COMPRISING PHOSPHOLIPIDS

(76) Inventors: Andrew Brown, Victoria (AU); Michelle Rowney, Victoria (AU)

(21) Appl. No.: 12/673,300

(22) PCT Filed: Aug. 15, 2008
(86) PCT No.: PCT/AU08/01191

§ 371 Date: Jan. 14, 2011

(30) Foreign Application Priority Data
Aug. 17, 2007 (AU) 2007904444

(51) Int. Cl. A61K 31/683; A61K 8/55; A61P 29/00; A61P 35/00; A61P 25/00; C12P 9/00; A23C 9/20; A23C 9/12; A23C 7/04

(52) U.S. Cl. 514/121; 435/131; 426/655; 426/2; 426/42

(57) ABSTRACT

The present invention provides compositions comprising phospholipids and particularly those comprising at least 40% phospholipid and at least 80% phospholipid as a percentage of total fat in the extract, comprising polyunsaturated and saturated phospholipids, in a ratio of saturated phospholipid to monounsaturated to polyunsaturated phospholipid of about 6:3:1 respectively, or comprising at least 40% phospholipid and less than 40% protein and methods for their production from dairy products.

The present invention relates to compositions comprising phospholipids and particularly phospholipid enriched dairy extracts and methods for preparing them.

### BACKGROUND

Phospholipids have been shown to have a number of health benefits, including liver protection, protection against tumour growth and memory improvement. The phospholipid sphingomyelin is required for cellular signalling, and has been shown to be involved in the control of cell proliferation, apoptosis, inflammation, and cancer. Sphingomyelin also inhibits intestinal absorption of cholesterol and fat in rats. Additionally phospholipids have been to shown to have good emulsification properties and have been used for the production of emulsions for drug delivery in the medical and cosmetic fields. They are also used in the production of liposomes.

The identification of these functions of phospholipids, in particular sphingomyelin, has led to increasing interest in techniques for isolating phospholipid fractions.

Phospholipids of interest are commonly found in the cell membrane, brain and neural tissue, retina and within some genera of microbes. All are impractical sources for lipid isolation.

Whole milk contains approximately 0.035% phospholipid, of which approximately 35% is in the milk serum, and the remaining 65% is in the milk fat globule membrane (MFGM). Buttermilk contains 0.13% phospholipid. The MFGM phospholipids are primarily phosphatidyl choline (PC), phosphatidyl ethanolamine (PE), and sphingomyelin (SM), with small amounts of phosphatidyl serine and phosphatidyl inositol (PI).

Milk products from waste dairy streams, such as buttermilk, provide a good starting material for phospholipid isolation and techniques for isolating phospholipids from these sources have been examined. These techniques range from traditional methods using solvent extraction to emerging technologies such as microfiltration and supercritical fluid extraction.

Solvent extraction of phospholipids is not desired as this required the use of toxic solvents.

Astaire, J. C. et al., (J. Dairy Sci. 2003 86:2297-2307) describe the concentration of polar milk fat globule membrane lipids from buttermilk by microfiltration using a membrane of 0.8μ pore size to concentrate the polar lipids and supercritical fluid extraction with carbon dioxide to remove exclusively non-polar lipids. The method described provides a concentrated phospholipid and protein mixture.

Corredig, M. et al., (J. Dairy Sci. 2003 86:2744-2750) describes microfiltration of buttermilk though a 0.1μ pore size membrane (cut off 250,000-500,000 Da) after addition of sodium citrate.

WO 02/34062 A1 in the name NV Marc Boone describes a method for obtaining products enriched in phospho- and sphingolipids using ultrafiltration over a membrane with a cut off ranging from 5,000-20,000 Da.

Morin, P. et al., (J. Dairy Sci. 2004 87:267-273) studies the effect of temperature and pore size on the separation of proteins and lipids during microfiltration of fresh or reconstituted buttermilk.

Because of the numerous uses known in the art for phospholipids it is desirable to provide phospholipid enriched products from economically viable sources.

### SUMMARY

In a first aspect the invention provides a composition comprising at least 40% phospholipid and at least 80% phospholipid as a percentage of total fat in the composition.

In a second aspect the invention provides a composition comprising polyunsaturated and saturated phospholipids, which phospholipids are present in the composition in a ratio of saturated phospholipid to monounsaturated phospholipid to polyunsaturated phospholipid of about 6:3:1 respectively.

In a third aspect the invention provides a composition comprising at least 40% phospholipid and less than 40% protein.

In a preferred embodiment of the third aspect the protein is hydrolysed. Preferably the protein is hydrolysed by an enzyme, particularly a protease. Preferred proteases are those falling within International class EC 3.4.21.62.

In an embodiment of the preceding aspects the composition is derived from a dairy product.

The inventors propose that dairy products can provide a commercially viable source of phospholipids and have devised a process for enriching dairy products for phospholipids as a percentage of total fat. Such enriched extracts find utility in all applications for which individual or mixtures of phospholipids have been proposed in the art.

In a fourth aspect the invention provides a method for preparation of a phospholipid enriched extract from a dairy product, the method comprises:
(a) contacting the milk product with a protease under appropriate conditions to allow hydrolysis of milk proteins to occur to produce a hydrolysate; and
(b) subjecting the hydrolysate to a filtration step to separate it into a retentate fraction and a permeate fraction, whereby phospholipids are enriched in the retentate fraction and at least some protein is present in the permeate fraction.

Phospholipids in an aqueous solution generally exist as a micelle, which behaves like a molecule with a molecular weight above 50 kDa, as do many proteins. The prior art methods of producing phospholipid enriched products using filtration use membranes which have a cut off that retains the phospholipids but also retains the proteins and thus provide mixtures which contain both protein and phospholipid. By introducing a hydrolysis step the protein is broken down to peptides of a size that is able to pass through the filter with other contaminants. This allows the method of the invention to prepare a more concentrated phospholipid enriched product than prior art methods.

The invention in a fifth aspect provides a phospholipid enriched dairy extract obtainable or obtained by the method of the fourth aspect of the invention.

The invention in a sixth aspect provides the use of a composition according to the first, second or third aspects of the invention or a phospholipid enriched diary extract according to the fifth aspect of the invention as a nutraceutical, pharmaceutical, cosmetic ingredient, food, food additive or functional food or as a starting material for the production of liposomes.

In a seventh aspect the invention provides a nutraceutical, pharmaceutical, cosmetic ingredient, food, food additive or functional food or starting material for production of liposomes comprising a composition according to the first, second or third aspects of the invention or a phospholipid enriched extract according to the fifth aspect of the invention.

In an eighth aspect the invention provides a pharmaceutical composition comprising a composition according to the first, second or third aspects of the invention or a phospholipid enriched extract according to the fifth aspect of the invention, and a pharmaceutically acceptable carrier.

The invention in a ninth aspect provides a method of treating disorders involving abnormal cellular signalling or cell proliferation, apoptosis, inflammation, cancer, or promoting memory improvement comprising administering an effective amount of a composition according to the first, second or third aspects of the invention or a phospholipid enriched extract according to the fifth aspect of the invention.

The invention in a tenth aspect provides for use of a composition according to the first, second or third aspects of the invention or an extract according to the fifth aspect of the invention, in the manufacture of a medicament for treating disorders involving abnormal cellular signalling or cell proliferation, apoptosis, inflammation, cancer for memory improvement or for production of emulsions for drug delivery in the medical and cosmetic fields or in the production of liposomes.

### DETAILED DESCRIPTION

The inventors have recognised the need for a commercially viable source of phospholipids and a process which allows the preparation of a phospholipid enriched extracts in an efficient manner. The inventors provide a method for providing a phospholipid enriched extract from dairy products, satisfying criteria such as phospholipid content of at least 40%, phospholipid as percentage of total fat as at least 80% or ratio of saturated to monounsaturated to polyunsaturated phospholipids of 6:3:1 or thereabouts or a ratio of total phospholipid to protein of at least 1:1, preferably 1.2:1, 1.5:1, 1.8:1 or 2:1. Such enriched fractions may be produced using a process wherein a dairy product is subjected to a protease and filtration to remove at least some of the milk protein in a permeate, whereby the retentate is enriched with phospholipid. Persons skilled in the art would be aware that the composition of the product of the process could be mimicked by combining the essential components obtained by other means.

A composition according to the first, second, third or fifth aspects of the invention having a phospholipid content of at least 40% may comprise:

1. a ratio of about 3:1 phosphatidyl choline to phosphatidyl inositol;

2. a ratio of about 1:1 phosphatidyl choline to phosphatidyl ethanolamine;

3. a phospholipid composition comprising at least one of phosphatidyl choline, phosphatidyl serine, phosphatidyl inositol, phosphatidyl ethanolamine, and sphingomyelin or combinations thereof;

4. roughly equal amounts of c18:1 cis to c16 fatty acids;

5. less than 2% lactose, preferably less than 1% lactose;

6. one or more gangliosides selected from GM3, GM2, GD3, GD2 and GD1b;

7. phosphatidyl choline at 20-30% of total phospholipids, preferably 25-27%;

8. phosphatidyl inositol at 7-10% of total phospholipids, preferably 8-9%;

9. phosphatidyl serine at 10-15% of total phospholipids, preferably 11-12%;

10. phosphatidyl ethanolamine at 25-30% of total phospholipids, preferably 27-28%;

11. sphingomylein at 15-20% of total phospholipids, preferably 18-19%; or

12. a typical fatty acid profile of c18:1 cis 25%±5%; c16 25±5%; c18 10.0%±2%; c14 8%±2% and c18:2%6 cis 5%±2% (where each fatty acid is expressed as a percentage of total fatty acids).

The composition may comprise at least 5, 10, 15, 20, 25, 30, 35 or 40% protein. The composition may comprise a ratio of total phospholipid to protein of at least 1:1, preferably 1.2:1, 1.5:1, 1.8:1 or 2:1. The protein component may comprise at least one hydrolysed dairy protein. The protein may be hydrolysed using an enzyme, preferably a protease. Suitable enzymes are described below in relation to the fourth aspect of the invention and it will be readily apparent to persons skilled in the art that such enzymes could be used to produce a protein hydrolysate which could be added to individual or a mixture of phospholipids to produce a phospholipid composition which mimics the essential features of that provided according to the method of the fourth aspect of the invention.

The composition may also include casein.

In relation to the fourth aspect of the invention, the term “phospholipid enriched” is intended to mean that the phospholipid:total protein ratio present in the extract is increased relative to the ratio present in the dairy product before the process is carried out.

For the extract to be considered phospholipid enriched, it should have a phospholipid content of at least 30% w/w, preferably at least 40% w/w and even more preferably at least 50% w/w. As a percentage of the total fat of the retentate the enriched extract may contain at least 80, 90 or 95% w/w of the fat as phospholipid.

The enrichment process preferably reduces the amount of protein present in the retentate by approximately one-third to one-quarter, if not more. The amount of ash and lactose are preferably also significantly reduced.

As used herein, the term “extract” refers to a partially purified portion of the dairy product.

Use of the term “efficient” is taken to mean an inexpensive and quick process when compared to methods which are currently employed to make phospholipid products. In one embodiment the method is particularly efficient as it can be carried out on one piece of plant apparatus. However this is not essential in the claimed method. It would be possible to carry out the hydrolysis step at a separate location to the filtration step and these need not be carried out sequentially, although this is preferred. The hydrolysate may be stored prior to the filtration step being commenced.

It will be apparent to those skilled in the art that the dairy product used as starting material in the method of the fourth aspect of the invention may be obtained from any lactating animal, e.g. ruminants such as cows, sheep, buffalos, goats, and deer, non-ruminants including primates such as a human, and monogastrics such as pigs. The dairy product may include buttermilk, cream, colostrum, milk fat globule membrane (MFGM), AMF serum, whey and whole milk or processed products made therefrom provided the processing does not include removal of phospholipids. AMF serum, a by product of the anhydrous milk fat (AMF) production process is a preferred milk product, especially when derived from cream or whey cream.

The protease used in the present invention may be any protease capable of cleaving peptide bonds in proteins.

Preferably the protease is an endoprotease. As the phospholipid enriched extract may be used in foodstuffs it is preferred that the protease is “food grade”, that is it is non-toxic over a broad range of concentrations and is tolerated when ingested by a subject.

The protease may have broad specificity so that all proteins in the dairy product are hydrolysed. Alternatively a mixture of proteases may be used, to provide broader specificity. One suitable protease is trypsin. Preferred proteases fall within the international class EC 3.4.21.62. These are subtilisin-type proteases which have broad specificity for peptide bonds, with a preference for a large uncharged residue in P1. Proteases falling within this class include Alcalase; Alcalase 0.6L; Alcalase 2.5L; ALK-enzyme; bacillopeptidase A; bacillopeptidase B; Bacillus subtilis alkaline proteinase Bioprase; Bioprase AL 15; Bioprase APL 30; Colistinase; subtilisin J; subtilisin S41; subtilisin Sendai; subtilisin GX; subtilisin E; subtilisin BL; Genenase I; Esperase; Maxatase; Thermoase PC 10; protease XXVII; Thermoase; Superase; subtilisin DY; subtilopeptidase; SP 266; Savinase 8.0L; Savinase 4.0T; Kazusase; protease VIII; Opticlean; Bacillus subtilis alkaline proteinase; Protin A 3L; Savinase; Savinase 16.0L; Savinase 32.0 L EX; Orientase 10B and protease S. Other proteases which may be useful include S Amano and P Amano, Umamizyme (all Amano Enzymes), Trypsin PTN and Alcalase 2.4L FG (both Novozyme).

Appropriate conditions to allow hydrolysis to occur will vary with the enzyme used. The optimum pH and temperature are closely related to the enzyme and changing the enzyme will change these other parameters. Optimum pH would generally be in the range 2.5-10, more likely pH 6.0 to 9.0 and most likely 8.0 or 9.0. Optimum temperature would generally be in the range 25 to 80° C., more likely 40 to 65° C. and most likely 50 or 60° C.

In a particular embodiment, as described in the examples, enzyme is Alcalase, the pH is pH 9 and the temperature is 50° C. One factor affecting the temperature is the heat tolerance of the membrane. The membrane used in the examples is not heat tolerant over 50° C. However, other membranes of higher heat tolerance could be used if the optimum temperature of the enzyme was higher.

The pH of the dairy product may be raised using any material of high pH. Suitable candidates include sodium or potassium hydroxide, although other hydroxides are also contemplated.

The optimal conditions for some of the suitable proteases are as follows:

| Protease | Optimum Temp ° C. | Optimum pH |
|---|---|---|
| Alcalase | 55-70 | 6.5-8.5 |
| Alcalase 0.6L | 55-70 | 6.5-8.5 |
| Alcalase 2.4L FG | 55-70 | 6.5-8.5 |
| Alcalase 1.5 FG | 55-70 | 6.5-8.5 |
| Bacillus subtilis alkaline | 55-70 | 6.5-8.5 |
| proteinase | | |
| bacillopeptidase A | 55-70 | 6.5-8.5 |
| Bioprase APL 30 | 60 | 5.5 |
| Esperase | 55-75 | 7.5-10.0 |
| Opticlean | 45-50 | 8-10 |
| Optimase | 45-50 | 8-10 |
| P Amano | 45 | 8 (7-8) |
| protease XXIV | 55-70 | 6.5-8.5 |
| proteinase bioprase | 60 | 5.5 |
| S Amano | 70 (65-70) | 8 (7-9) |
| Savinase | 45-50 | 9-11 |
| Savinase 16.0L | 45-50 | 9-11 |
| Savinase 32.0 L EX | 45-50 | 9-11 |
| Savinase 4.0T | 45-50 | 9-11 |
| Savinase 8.0L | 45-50 | 9-11 |
| subtilisin E | 50 | |
| subtilisin GX | 40-55 | |
| subtilisin S41 | 40-55 | 7-8.5 |
| subtilisin Sendai | | 10 |
| Subtilopeptidase | 55-70 | 6.5-8.5 |
| Thermoase | 65-70 | 7-8.5 |
| Thermoase PC 10 | 65-70 | 7-8.5 |
| Umamizyme | 45-50 | 6-8 |
| Trypsin | 40-50 | 7.5-8.5 |

As used herein, the term “hydrolysis” refers to the breakdown of proteins or polypeptides into shorter polypeptides, and oligopeptides and possibly, to a small extent, component amino acids by cleavage of one or more peptide bonds joining the constituent amino acids.

Endoproteases cleave the peptide bonds within a protein and exoproteases degrade the protein molecules from one end.

A milk protein is classed as “hydrolysed” or hydrolysis has occurred if at least some of the protein is hydrolysed into smaller fragments. The protein need not be broken down into constituent amino acids to be classified as hydrolysed.

Accordingly, the term “hydrolysis” is intended to encompass at least partial hydrolysis of a milk protein. In one embodiment the net degree of hydrolysis (%) is 0.5%, 1%, 2%, 3%, 4%, 5%, 10%, 15% 20%, 25%, 30%, 35%, 40%, 45%, 50%, 55%, 60%, 65%, 70%, 75%, 80%, 85%, 90%, 95%, 99% or 100%.

The term “hydrolysate” refers to the mixture of intact proteins or polypeptides, shorter polypeptides, and oligopeptides and component amino acids which is produced by hydrolysis.

The term “permeate” refers to the fraction which has passed through or permeated the intact membrane.

The term “retentate” refers to the fraction which is retained by the membrane.

Reference to “at least some protein is present in the permeate” is intended to mean that at least some of the protein present in the milk product has been able to permeate the membrane.

Reference to “protein” includes oligo-peptides, peptides and amino acids.

The membrane used for the filtration step in the method of the fourth aspect of the invention is preferably a microfiltration (MF) membrane. Any membrane with a nominal molecular weight cut-off (NMWCO) of greater than 5 kDa may be suitable, although a higher NMWCO of a least 20 kDa, 30 Kda, 40 kDa, 50 kDa, 60 kDa, 70 kDa, 80 kDa, 90 kDa or 100 kDa would be better. The pore size of the membrane (and hence size cut off) should be balanced with operating pressure to allow retention of phospholipids in the retentate. Membranes with lower cut-offs would require higher pressures, but the flow rate through the membrane would probably be lower.

The examples show a membrane with a nominal pore size of 0.3 μm (Desal JX 0.3 μm MF) operating at 2 Bar pressure. The maximum permissible membrane porosity is of the order of 4-5 μm, which corresponds to the size of a phospholipid micelle and the largest commercially available MF membranes and is far bigger than a single protein, for which the concept of NMWCO is relevant. The cross-membrane pressure should be 2 Bar or less. The upper limit will depend on the membrane being used but increasing the pressure will result in more phospholipids passing through the membrane into the permeate.

It is anticipated that the lower the pore size the greater proportion of phospholipid is retained in the retentate. A pore size of around 0.1 μm (0.05 to 0.1, 0.2, 0.3, 0.4 or 0.5 μm) appears optimal, with a pore size of 0.8 μm allowing too much phospholipid to pass into the permeate.

An embodiment of the fourth aspect includes an initial filtration step prior to step (a) to enrich the milk product for phospholipids and optionally remove soluble contaminants such as lactose, ash (inorganic compounds, such as salt and metal ions) and whey proteins, particularly those contaminants that may affect the activity of the protease. If this initial filtration step is performed the method of the invention is carried out on the retentate of the initial filtration step.

The addition of the initial filtration step reduces the amount of protease required and increases the by-product stream.

Once the volume of the milk product in the retentate is reduced by the initial filtration step, preferably to 20-25% of the original volume, extra water may be added to the retentate and filtration (diafiltration with water) continued to remove further soluble contaminants from the retentate.

Prior to contacting the milk product or its retentate with protease removal of fluid may be stopped or paused and the conditions altered to be appropriate for hydrolysis of milk proteins. This may involve altering the pH and/or temperature. In a particular embodiment the pH is increased to 9.0 and the temperature increased to 50° C.

The protease is added in an amount and for a sufficient time to allow an appropriate amount of hydrolysis. In a particular embodiment 30 mL Alcalase 2.4L FG is added to 40 L of milk product or retentate (i.e. 0.075% v/v) but it is envisaged that much less could be used, down to as little as 0.05%, 0.01% or 0.001% v/v depending on the enzyme activity. The maximum amount used may be 5% v/v, although this amount would be uneconomic, irrespective of the enzyme being used. The realistic maximum is sensibly 1% for most enzymes, but this is more related to economy rather than performance. Persons skilled in the art would readily be able to determine a suitable concentration of protease to use, depending on its activity.

In a particular embodiment the hydrolysis step is performed for 1 to 2 hours, and particularly 1.5 hours. This can varied depending on the enzyme activity.

After the hydrolysis step the pH may be measured and if it is not the desired final pH of the retentate it may be adjusted. Generally the desired final pH is pH 6.5 to 7.5. If necessary the pH may be adjusted using an acid such as hydrochloric acid.

The filtration of the hydrolysate is preferably diafiltration with water. Filtration is continued until the permeate contains minimal or no solids (i.e. is estimated to be 0.0 Brix with a refractometer.

The protease may be deactivated by heating to denaturing temperature for a short period. For Alcalase, deactivation is achieved by heating to in excess of 85° C. for about 10 minutes. Whilst it is envisaged that the protease will be removed from the retentate during the filtration step, the deactivation step may be necessary for regulatory approval if the retentate is to be used in foods or nutraceuticals.

The retentate may be dried or cooled for storage. Suitable drying methods include freeze drying or spray drying.

Using the method of the invention on AMF serum with an initial filtration step and hydrolysis with Alcalase produces a retentate enriched for phospholipids, with over 80% phospholipids as a percentage of the total fat of the retentate. The retentate is also enriched for cholesterol. The cholesterol may be removed from the first retentate or the hydrolysate by methods known in the art, for example using a cholesterol-binding compound, such as a cyclodextrin. In a particular embodiment cholesterol is removed after the hydrolysis step.

The membrane used for filtration of the hydrolysate may be the same or different from the membrane used in the initial filtration step. In a particular embodiment the membrane and conditions used in the initial filtration step are the same as for the filtration of the hydrolysate, thus allowing the enrichment for phospholipids to be performed in one piece of plant.

The process according to the fourth aspect of the invention may be performed in isolation to prepare a phospholipid enriched extract, or may be incorporated as part of an integrated fractionation process in which other desired milk product components are fractionated.

Use of the term “product”, “composition” or “extract” is not intended to limit the invention to the production of phospholipid enriched end products or extracts. The phospholipid enriched extract produced by the method of the invention may be used as a starting or intermediate product in the production of other products.

A method according to a particular embodiment of the invention is described at Example 1.

Since phospholipids are involved in a number of physiological functions, their preparation using the process according to the invention provides an ideal and economical source of phospholipids which can subsequently be directed towards these functions. For example the composition or phospholipid enriched extract produced by the method of the present invention may be used in the production of nutraceuticals, pharmaceuticals, cosmetics, foods and liposomes.

The term “nutraceutical” as used herein refers to an edible product isolated or purified from food, in this case from a dairy product, which is demonstrated to have a physiological benefit or to provide protection or attenuation of an acute or chronic disease or injury when orally administered. The nutraceutical may thus be presented in the form of a dietary preparation or supplement, either alone or admixed with edible foods or drinks. The nutraceutical may have positive clinical effect on memory or disorders involving abnormal cellular signalling or cell proliferation, apoptosis, inflammation and cancer, it may have a protective effect on the liver and may inhibit intestinal absorption of cholesterol and fat.

The nutraceutical composition may be in the form of a soluble powder, a liquid or a ready-to-drink formulation. Alternatively, the nutritional composition may be in solid form; for example in the form of a ready-to-eat bar or breakfast cereal. Various flavours, fibres, sweeteners, and other additives may also be present.

The nutraceutical preferably has acceptable sensory properties (such as acceptable smell, taste and palatability), and may further comprise vitamins and/or minerals selected from at least one of vitamins A, B1, B2, B3, B5, B6, B11, B12, biotin, C, D, E, H and K and calcium, magnesium, potassium, zinc and iron.

The composition may be fed to a subject via a nasogastric tube, jejunum tube, or by having the subject drink or eat it.

The nutraceutical composition may be produced as is conventional; for example, the composition may be prepared by blending together the composition or phospholipid enriched extract and other additives. If used, an emulsifier may be included in the blend. Additional vitamins and minerals may be added at this point but are usually added later to avoid thermal degradation.

If it is desired to produce a powdered nutraceutical composition, the composition or phospholipid enriched extract may be admixed with additional components in powdered form. The powder should have a moisture content of less than about 5% by weight. Water, preferably water which has been subjected to reverse osmosis, may then be mixed in to form a liquid mixture.

If the nutraceutical composition is to be provided in a ready to consume liquid form, it may be heated in order to reduce the bacterial load. If it is desired to produce a liquid nutraceutical composition, the liquid mixture is preferably aseptically filled into suitable containers. Aseptic filling of the containers may be carried out using techniques commonly available in the art. Suitable apparatus for carrying out aseptic filling of this nature is commercially available.

The composition or phospholipid enriched extract may also be provided as a food, a food additive or functional food.

The composition or phospholipid enriched extract may also be formulated in a pharmaceutical composition suitable for administration to a subject.

Preferably the pharmaceutical composition also comprises one or more pharmaceutically acceptable carriers, diluents or excipients. Such compositions may comprise buffers such as neutral buffered saline, phosphate buffered saline and the like; carbohydrates such as glucose, mannose, sucrose or dextrans; mannitol; proteins; polypeptides or amino acids such as glycine; antioxidants; chelating agents such as EDTA; adjuvants and preservatives. Compositions of the present invention may be formulated for intravenous administration, topical application or oral consumption.

Such a composition may be administered to a subject in a manner appropriate to the disease to be treated and/or prevented. The quantity and frequency of administration will be determined by such factors as the condition of the subject and the type and/or severity of the subject's disease. Appropriate dosages may also be determined by clinical trials. An effective amount of the composition can be determined by a physician with consideration of individual differences in age, weight, disease severity, condition of the subject, route of administration and any other factors relevant to treatment of the subject. Essentially, an “effective amount” of the composition is an amount which is sufficient to achieve a desired therapeutic effect.

In another aspect, the present invention provides methods for the treatment and/or prevention of diseases. Such treatment methods comprise administering to a subject an effective amount of a composition, nutraceutical or pharmaceutical composition as described above. Such administration may treat or prevent any disease or disorder in which increased phospholipids are advantageous. Suitable patients include those desiring memory improvement or requiring treatment for disorders involving abnormal cellular signalling or cell proliferation, apoptosis, inflammation, and cancer.

In a further aspect the composition or phospholipid enriched extract may be used in the production of emulsions for drug delivery in the medical and cosmetic fields or in the production of liposomes. Such liposomes may be useful for drug delivery and in the production of cosmetics, such as skin creams.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

It must also be noted that, as used in the subject specification, the singular forms “a”, “an” and “the” include plural aspects unless the context clearly dictates otherwise.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

The invention is now further described in detail by reference to the following examples. These examples are provided for purposes of illustration only, and are not intended to be limiting unless otherwise specified. Thus, the invention encompasses any and all variations which become evident as a result of the teaching provided herein.

### EXAMPLES

In the tables in the examples that follow the following abbreviations are used:
DF—diafiltration
RET—retentate
HYD—hydrolysis
PERM—permeate
MF—microfiltration
NA, result not available (due to lack of material for testing or lack of suitability for testing)
NMR, nuclear magnetic resonance;
PL, phospholipids;
2LPC, 2-lysophosphatidylcholine;
2LPS, 2-lysophosphatidylethanolamine;
2LPE, 2-lysophosphatidylserine;
DHSM, dihydrosphingomyelin;
PC, phosphatidylcholine;
PE, phosphatidylethanolamine;
PI, phosphatidylinositol;
PMG, phosphonomethyl glycine;
PS, phosphatidylserine;
SM, sphingomyelin.

### Example 1

### Process for Preparation of a Phospholipid Enriched Fraction from AMF Serum Using Alcalase 2.4 FG

AMF serum is a by-product of the AMF (anhydrous milk fat) manufacturing process and maybe manufactured from full cream milk or whey. This process involves several steps, although most steps involve a passage through a separator. A separator is a machine containing a series of rapidly spinning discs, which cause the incoming liquid to spin. The spinning imposes a centrifugal force (5,000-10,000 g), which results in the formation of two phases based on the difference in the specific gravity (the heaviest phase is pushed outwards and the lightest phase collects in the middle).

Full cream milk is heated to approximately 55° C. and separated by a passage through a separator, which leads to the formation of phospholipid-reduced skim milk and phospholipid enriched choice cream. Full cream milk may also be used to manufacture cheese and the soluble fraction draining from the cheese is known as whey. Whey may also be passed through a separator at approximately 55° C., which leads to the formation of phospholipid-reduced whey and phospholipid enriched whey cream. From this point choice cream and whey cream are processed identically and maybe pooled for further processing. If not already warm, the cream is then reheated and separated a second time, which results in a phospholipid-reduced concentrated fat phase and a phospholipid enriched aqueous phase (buttermilk). The buttermilk is then separated a third time, which results in a phospholipid-reduced concentrated fat phase and an aqueous phase (AMF serum) with a greater proportion of phospholipids than present in buttermilk. The AMF serum is then cooled and stored for further processing. The composition of AMF serum is shown as (A) in Table 1.1.

The AMF serum produced by this or any other method is then subjected to a phospholipid enrichment method according to the first aspect of the invention.

The minimum apparatus for this process are a plate heat-exchanger, a vat and a pump generating pressure across a pair of filtration membranes. The fluid circulates continually throughout the process to ensure mixing. Table 1.1 relates to this process and gives an indication of the composition of each fraction (capital letter in the text below relates to the capital letter in the first row of Table 1.1).

1. The AMF serum is microfiltrated (0.3 μm membranes, 2 Bar) and phospholipids remain in the retentate. The volume is reduced until approximately 20-25% of the original liquid remains. Lactose, ash (inorganic compounds, such as salt and metal ions) and whey proteins permeate (B) across the membrane and are sent to an alternative process. The temperature should be 10° C. to maintain product quality and the pH is uncontrolled.
2. Extra water is added to the retentate and step 1 is continued (i.e. diafiltrate with water). The aim of this step is further removal of soluble contaminants from the retentate (C).
3. Stop removing fluid by MF, but continue circulation to ensure mixing.
4. Concurrently add 2M (8%) NaOH until the retentate is pH 9.0 and heat until the temperature is 50° C.
5. Add Alcalase 2.4L FG (an endoprotease. 30 mL to 40 L) to the retentate and maintain the conditions specified in step 3 until the pH stops decreasing. Extra NaOH may be periodically required to increase the pH. The optimum hydrolysis period is one-and-a-half hours at the stated rate of enzyme addition.
6. If the pH is not the desired final pH, adjust to the specified pH (6.5-7.5) by the addition of an acid (ideally HCl, but others may be suitable).
7. Start adding water and recommence MF (i.e. diafiltrate with water to remove non-phospholipid components). The retentate contains all the phospholipids and is the PLRME (E). The permeate (D) is composed of Alcalase/peptides/ash/lactose and can be sent to an alternative process. The diafiltration step is continued until the permeate contains undetectable levels of contaminants (i.e. estimated to be 0.0 Brix with a refractometer).
8. Deactivate the Alcalase by heating to 85° C. for 10 min.
9. If desirable dry (any type of drying normally used for dairy products would be suitable, freeze-drying or spray drying would be especially desirable) or cool to refrigerated temperatures for storage.

TABLE 1.1
Composition of powders derived from AMF serum by MF and hydrolysis.

| | | | | D. | E. |
|---|---|---|---|---|---|
| | | B. | C. | AMF serum | AMF serum |
| | A. | AMF serum | AMF serum | MF RET/ | MF RET/ |
| COMPONENT | AMF serum | MF PERM | MF RET | HYD/PERM | HYD/RET |
| Ash (%) | 6.8 | 9.1 | 4.5 | 6.5 | 5.9 |
| Moisture (%) | 2.2 | 2.4 | 1.9 | 3 | 1.1 |
| Fat (%) | 14.2 | <0.1 | 32 | 0.95 | 57.1 |
| Phospholipids (%) | 10.2 | 0.4 | 15 | <0.1 | 47.9 |
| Non-PL fat (%) | 4 | 0 | 17 | 0.95 | 9.2 |
| Cholesterol | 130 | <1 | 320 | <1 | 610 |
| (mg/100 g) | | | | | |
| Lactose (%) | 47.9 | 84.6 | 1 | 1.8 | 0.7 |
| Protein (%) | 26.7 | 4.48 | 56.6 | 85.4 | 27.6 |
| Phospholipids | 71.8 | — | 46.9 | <10 | 83.9 |
| (% total fat) | | | | | |

### Process Variations

The following variations may occur without substantially altering the end product.

1. Step 1 and/or 2 may be omitted entirely. The result with degradation of the by-product stream and an increase in the protease required.
2. The protease may be changed, as described below (examples 2 and 9). Changing the protease will alter the temperature and pH optimums.
3. Step 8 may be omitted.

### Example 2

### Process for Preparation of a Phospholipid Enriched Fraction from Buttermilk and AMF Serum Derivatives Using S Amano and P Amano

### Raw Materials

The buttermilk (92 kg) and AMF serum (80 kg) used were collected from Rochester in 10 L drums and transported immediately to Cobram. The buttermilk was placed in the cool room on arrival. The AMF serum was found to be 45.0° C. on arrival at Cobram and was cooled to 30.5° C. over approximately 2 h prior to membrane filtration.

### Membrane Filtration Methodology

The membrane filtration plant (Model 92 Laboratory Unit, Filtration Engineering Co. Inc.) was fitted with Desal JX (0.3 μm MF) membranes for all experiments. All MF was undertaken at 0 Bar (no valve 2 closure) and collection of retentate commenced only after permeate composition had equilibrated. The recorded data for pre- (Tables 2.1 and 2.2) and post-hydrolysis (Tables 2.3 and 2.4) are presented below.

TABLE 2.1
Details of the MF concentration and
2 × diafiltration of AMF serum.

| | Starting | Permeate | DF water | Temp |
|---|---|---|---|---|
| Time | mass (kg) | removed (kg) | added (L) | (° C.) |
| 2:35 | 80 | 63.8 (4 brix) | 20 | 30.5 |
| 4:10 | | | | 31.5 |
| 4:50 | | 20 | 20 | 24 |
| 5:30 | | 20 (0.6 brix) | 0 | |

Final Brix of retentate in tank: 18.5
Mass/concentration of retentate for hydrolysis: 23.05 kg/7 Brix

TABLE 2.2
Details of the MF concentration and 3 × diafiltration of buttermilk.

| Starting | Permeate | DF water | Temp |
|---|---|---|---|
| mass (kg) | removed (kg) | added (L) | (° C.) |
| 91.43 (6.5 brix) | 75.0 (4 brix) | 10 | 8.6 |
| | 17.89 | 10 | |
| | 19.72 (0.8 brix) | 10 | |
| | 18.87 (0.2 brix) | 0 | |

Final Brix of retentate: 12.5
Mass/concentration of retentate for hydrolysis: 14.35 kg/9 Brix

TABLE 2.3
Details of the MF concentration and 2 ×
diafiltration of AMF serum hydrolysate.

| Starting | Permeate | DF water | Temp |
|---|---|---|---|
| mass (kg) | removed (kg) | added (L) | (° C.) |
| 22.96 (5.5 brix) | 9.5 (2.5 brix) | 20 | Cool |
| | 19.5 | 20 | |
| | 24.5 (0.2 brix) | 0 | |

Final Brix of retentate: 6.0

TABLE 2.4
Details of the MF concentration and 2 ×
diafiltration of buttermilk hydrolysate.

| Starting | Permeate | DF water | Temp |
|---|---|---|---|
| mass (kg) | removed (kg) | added (L) | (° C.) |
| 15.8 (5.5 brix) | 3.83 (3.0 brix) | 20 | Cool |
| | 19.3 | 40 | |
| | 43.9 (0.2 brix) | 0 | |

Final Brix of retentate: 4.0

### Hydrolysis Method

The MF retentates (Tables 2.1 and 2.2) were heated to 45° C. and hydrolysed with a mixture of proteases S Amano and P Amano (buttermilk 1.94 g of both enzymes, AMF serum 2.5 g of both enzymes) for 2 h. The hydrolysis mixtures were continuously agitated and the pH was maintained within the range pH 6.9-7.3 by the addition of 4M NaOH (volume added during hydrolysis: buttermilk, 94 mL; AMF serum, 100 mL).

### Drying

All raw materials and retentate samples were freeze dried at 45° C. for 48 h. All permeate samples were freeze dried at 55° C. for 36 h.

### Results

The composition of the products derived from buttermilk and AMF serum are compared in Table 2.5. The results show that AMF serum is a superior source of phospholipids. The results also show that MF of the raw material leads to a dramatic rise in the phospholipids content by removing lactose, some proteins and a large proportion of the non-phospholipid lipid. Protein hydrolysis followed by MF also leads to an increase in the phospholipids by reducing the amount of protein present by approximately one-third to one-quarter. The presence of large amounts of residual protein after hydrolysis and MF is a disappointing outcome and may be due to an unsuitable choice of proteases (more aggressive protease may be better) or phospholipids preventing proteases gaining access to proteins (micelles may be the reason and homogenization may allow the proteases better access).

TABLE 2.5
Composition of powders derived from buttermilk and AMF serum by MF and hydrolysis.

| | | | Butter- | | | |
|---|---|---|---|---|---|---|
| | | Butter- | milk | | | AMF serum |
| | Butter- | milk | MF RET/ | | AMF serum | MF RET/ |
| COMPONENT | milk | MF RET | HYD/RET | AMF serum | MF RET | HYD/RET |
| Ash (%) | 7.4 | 5.9 | 9.2 | 6.3 | 6.2 | 7.5 |
| Moisture (%) | 2.5 | 0.7 | 2 | 2.3 | 1 | 1.7 |
| Fat (%) | 10.8 | 16.3 | 34.3 | 17.4 | 26.3 | 48.2 |
| Phospholipids (%) | 1.4 | 9.6 | 21.4 | 10.1 | 27.4 | 44.7 |
| Non-PL fat (%) | 9.4 | 6.7 | 12.9 | 7.3 | 0 | 3.5 |
| Cholesterol | 85 | 240 | 500 | 160 | 330 | 550 |
| (mg/100 g) | | | | | | |
| Lactose (%) | 49.3 | 1.9 | 0.5 | 45.2 | 4.3 | <0.3 |
| Protein (%) | 25.6 | 75.4 | 53.1 | 28.1 | 60.2 | 44.2 |
| Phospholipids | 13.0 | 58.9 | 62.4 | 58.0 | 104.2 | 92.7 |
| (% total fat) | | | | | | |
| Total nitrogen (%) | 4.0 | | | 4.4 | | |
| Casein Nitrogen (%) | 2.6 | | | 2.7 | | |
| Non-Casein | 1.4 | | | 1.7 | | |
| Nitrogen (%) | | | | | | |
| Whey Protein | 1.0 | | | 1.2 | | |
| Nitrogen (%) | | | | | | |
| Non-Protein | 0.4 | | | 0.5 | | |
| Nitrogen (%) | | | | | | |
| Fatty acid profile | | | | | | |
| (% fatty acids) | | | | | | |
| C: 4.0 | 4.2 | | | 3.3 | | |
| C: 6.0 | 2.1 | | | 1.7 | | |
| C: 8.0 | 1.3 | | | 1 | | |
| C: 10.0 | 3.2 | | | 2.5 | | |
| C: 10.1 ω1 cis | 0.3 | | | 0.2 | | |
| C: 12.0 | 3.9 | | | 3.1 | | |
| C: 12.1 ω9 cis | 0.1 | | | 0.1 | | |
| C 12.1 ω6 cis | <0.1 | | | <0.1 | | |
| C: 12.1 ω3 cis | 0.3 | | | 0.2 | | |
| C: 14.0 iso | 0.1 | | | 0.1 | | |
| C: 14.0 | 11.1 | | | 9.4 | | |
| C: 14.1 ω5 cis | 1.3 | | | 1 | | |
| C: 15.0 ante-iso | 0.5 | | | 0.1 | | |
| C: 15.0 | 1.3 | | | 1.1 | | |
| C: 16.0 iso | 0.2 | | | 0.2 | | |
| C: 15.1 ω5 cis | <0.1 | | | <0.1 | | |
| C: 16.0 | 27.7 | | | 25.8 | | |
| C: 16.1 trans | 0.3 | | | 0.3 | | |
| C: 16.1 ω7 cis | 1.5 | | | 1.4 | | |
| C: 17.0 iso | 0.4 | | | 0.4 | | |
| C: 17.0 ante-iso | 0.7 | | | 0.7 | | |
| C: 17.0 | 0.6 | | | 0.6 | | |
| C: 17.1 ω7 cis | 0.3 | | | 0.3 | | |
| C: 18.0 iso | 0.1 | | | 0.1 | | |
| C: 18.0 | 8.6 | | | 9.8 | | |
| C: 18.1 trans | 3.1 | | | 2.8 | | |
| C 18.1 cis | 19.1 | | | 23.1 | | |
| C: 18.2 trans | 0.6 | | | 0.5 | | |
| C: 18.2 cis9 tr12 | 0.7 | | | 0.7 | | |
| C: 18.2 tr9 cis12 | <0.1 | | | 0.3 | | |
| C: 18.2 ω6 cis | 2 | | | 3.5 | | |
| C: 18.3 trans | 0.1 | | | 0.1 | | |
| C: 18.3 ω6 cis | <0.1 | | | <0.1 | | |
| C: 18.3 ω3 cis | 0.9 | | | 1.2 | | |
| C: 20.0 | 0.1 | | | 0.1 | | |
| C: 18.2 conj | 1.1 | | | 1.2 | | |
| C: 18.4 ω3 | <0.1 | | | <0.1 | | |
| C: 20.1 ω9 cis | 0.1 | | | 0.1 | | |
| C: 20.2 ω6 cis | <0.1 | | | <0.1 | | |
| C: 20.3 ω6 cis | 0.1 | | | 0.3 | | |
| C: 20.4 ω6 cis | <0.1 | | | <0.1 | | |
| C: 20.3 ω3 cis | 0.2 | | | 0.5 | | |
| C: 22.0 | 0.1 | | | 0.1 | | |
| C: 20.4 ω3 | <0.1 | | | <0.1 | | |
| C: 22.1 ω9 cis | <0.1 | | | <0.1 | | |
| C: 20.5 ω3 cis | 0.1 | | | 0.3 | | |
| C: 22.2 ω6 cis | <0.1 | | | <0.1 | | |
| C: 21.5 ω3 | <0.1 | | | <0.1 | | |
| C: 22.5 ω6 | <0.1 | | | <0.1 | | |
| C: 24.0 | <0.1 | | | <0.1 | | |
| C: 24.1 ω9 cis | <0.1 | | | <0.1 | | |
| C: 22.5 ω3 | <0.1 | | | <0.1 | | |
| C: 22.6 ω3 cis | <0.1 | | | <0.1 | | |
| Others | 1.6 | | | 1.8 | | |

A MF membrane with pores of 0.3 μm has been shown to allow ash, lactose, proteins and peptides to be removed from a phospholipid-containing mixture, while retaining the phospholipids (Table 2.6). In this trial non-phospholipid lipids also appear to have passed through the MF membrane.

TABLE 2.6
Composition of permeate powders derived from
buttermilk and AMF serum by MF and hydrolysis.

| | | | Butter- | | AMF | AMF |
|---|---|---|---|---|---|---|
| | | Butter- | milk MF | | serum | serum MF |
| | Butter- | milk MF | RET/HYD/ | AMF | MF | RET/HYD/ |
| COMPONENT | milk | PERM | PERM | serum | PERM | PERM |
| Ash (%) | 7.4 | 8.8 | 5 | 6.3 | 8.7 | 7.1 |
| Moisture (%) | 2.5 | 1.9 | 4.9 | 2.3 | 1.8 | 2.2 |
| Fat (%) | 10.8 | <0.10 | 0.2 | 17.4 | <0.10 | 0.2 |
| Phospholipids (%) | 1.4 | <0.1 | <0.1 | 10.1 | <0.1 | 0.1 |
| Non-PL fat (%) | 9.4 | <0.10 | 0.2 | 7.3 | <0.10 | 0.1 |
| Cholesterol | 85 | <1 | 2 | 160 | <1 | 1 |
| (mg/100 g) | | | | | | |
| Lactose (%) | 49.3 | 85.2 | 3.4 | 45.2 | 86.4 | 6 |
| Protein (%) | 25.6 | 6.2 | 87.2 | 28.1 | 5.57 | 77.4 |

### Example 3

### Process for the Preparation of a Phospholipid Enriched Fraction from AMF Serum Using Umamizyme

### Raw Materials

The AMF serum (55.23 kg) used were collected from Rochester in 15 L drums and transported to Cobram.

### Membrane Filtration Methodology

The membrane filtration plant (Model 92 Laboratory Unit, Filtration Engineering Co. Inc.) was fitted with Desal JX (0.3 μm MF) membranes. All MF was undertaken at 0 Bar (no valve 2 closure) and collection of retentate commenced only after permeate composition had equilibrated. The recorded data for pre- (Table 3.1) and post-hydrolysis (Table 3.2) is presented below.

TABLE 3.1
Details of the MF concentration and
2 × diafiltration of AMF serum.

| Starting | Permeate | DF water | Temp |
|---|---|---|---|
| mass (kg) | removed (kg) | added (L) | (° C.) |
| 55.23 (7 brix) | 44.0 (3.2 brix) | 18 | Cool |
| | 16.0 | 40 | |
| | 36.66 (0.0 brix) | 0 | |

Final Brix of retentate: 9
Brix of retentate diluted prior to hydrolysis: 2.5
Mass/concentration of retentate for hydrolysis: not measured.

TABLE 3.2
Details of the MF concentration and trickle
diafiltration of AMF serum hydrolysate.

| Starting | Permeate | DF water | Temp |
|---|---|---|---|
| mass (kg) | removed (kg) | added (L) | (° C.) |
| 35 L (2.8 brix) | 26 (0.8 brix) | Commence trickle | 42 |
| | 25.0 (0.2 brix) | End trickle | 32 |
| | 10.0 | | |

Final Brix of retentate: 7 (pH 7.34 at 25.0° C. after cooling with chilled water coil)
Collected approximately 11 L without dilution and 6 L with some dilution

### Hydrolysis Method

The MF retentate (Table 3.1) was heated to 45° C. and hydrolysed with Amano Umamizyme (10.2 g) for 96 min. Hydrolysis was undertaken in the MF plant. The liquid was heated or cooled by a coil in the retentate tank. Water was added to the MF retentate present in the tank until the fluid covered the heating coil. The plant continued to operate throughout the hydrolysis to provide mixing. The pH was maintained within the range pH 7.0-8.3 by the addition of 4M NaOH. The relevant details recorded are presented in Table 3.3.

TABLE 3.3
Details AMF serum retentate hydrolysis.

| | | | pH | | NaOH |
|---|---|---|---|---|---|
| | | Temp | Pre | Post | added |
| Time | Comment | (° C.) | NaOH | NaOH | (g) |
| 1047 | Recirculating | 25.3 | 6.05 | — | — |
| 1100 | Commence heating | 25.9 | 5.99 | — | — |
| 1114 | Adjust pH (with NaOH) | 46.2 | 5.71 | 9.55 | 170 |
| 1122 | Commence hydrolysis | 45.2 | 8.38 | — | — |
| 1128 | Sampled for drying | — | — | — | — |
| 1136 | Adjust pH | 43.2 | 7.00 | 8.38 | 80 |
| 1205 | Monitor temp and pH | 42.8 | 7.52 | — | — |
| 1258 | Commence MF | 41.6 | 7.25 | — | — |

### Drying

All samples were freeze dried at 45° C. for 48 h.

### Results

Amano Umamizyme is a protease that can be used in the manufacture of a phospholipid enriched product (Table 3.4), but is an inferior enzyme for the production of the phospholipid enriched product, especially when compared to the phospholipid-enrich product obtained by hydrolysis with Alcalase 2.4L FG (Example 1), S Amano and P Amano (Example 2), or Trypsin (Example 9).

TABLE 3.4
Composition of powders derived from AMF serum by MF and hydrolysis.

| | | | | AMF serum | AMF serum |
|---|---|---|---|---|---|
| | | AMF serum | AMF serum | MF RET/ | MF RET/ |
| COMPONENT | AMF serum | MF PERM | MF RET | HYD/PERM | HYD/RET |
| Ash (%) | 6.7 | 9.7 | 4.2 | 7.7 | 3.8 |
| Moisture (%) | 4.4 | 4.9 | 5.8 | 7.4 | 2.0 |
| Fat (%) | 14.6 | 0.0 | 34.2 | 0.2 | 56.4 |
| Phospholipids (%) | 8.6 | <0.1 | 18.4 | <0.1 | 31.1 |
| Non-PL fat (%) | 6.0 | 0.0 | 15.8 | 0.2 | 25.3 |
| Cholesterol | 120 | <1 | 270 | <1 | 440 |
| (mg/100 g) | | | | | |
| Lactose (%) | 44.1 | 76.1 | 1.1 | 5.8 | 0.3 |
| Protein (%) | 27.1 | 5.5 | 52.2 | 74.4 | 36.8 |
| Phospholipids | 59.0 | 0.0 | 53.9 | 0.0 | 55.1 |
| (% total fat) | | | | | |

### Example 4

### Process for the Preparation of a Phospholipid Enriched Fraction from AMF Serum Derivatives Using Cyclodextrin and Alcalase

### Raw Materials

The AMP serum (54.34 kg) used were collected from Rochester in 15 L drums and transported to Cobram.

### Membrane Filtration Methodology

The membrane filtration plant (Model 92 Laboratory Unit, Filtration Engineering Co. Inc.) was fitted with Desal JX (0.3 μm MF) membranes and a plate cooler was fitted to the valve 5 outlet to allow heating/cooling of the retentate. All MF was undertaken at 0 Bar (no valve 2 closure) and collection of retentate commenced only after permeate composition had equilibrated. The recorded data for pre-(Table 4.1) and post-hydrolysis (Table 4.2) is presented below.

TABLE 4.1
Details of the MF concentration and diafiltration of AMF serum.

| Starting | Permeate | DF water | Temp |
|---|---|---|---|
| mass (kg) | removed (kg) | added (L) | (° C.) |
| 54.34 (7.2 brix) | 46.38 (3.5 brix) | 20 + trickle | 7-15 |
| | 36 | 0 | |

Final Brix of retentate: 13
Brix of retentate diluted prior to hydrolysis: 4
Mass/concentration of retentate for hydrolysis: not measured.

TABLE 4.2
Details of the MF concentration and 2 ×
diafiltration of AMF serum hydrolysate.

| Starting | Permeate | DF water | Temp |
|---|---|---|---|
| mass (kg) | removed (kg) | added (L) | (° C.) |
| 40 | 28.5 (3 brix) | Unknown | 47.5 |
| | | | 38.7-50.7 |
| | 29.0 (0.2 brix) | Unknown | 47.5 Adjust pH to 6.3 |
| | 26.38 | 0 | 12 |

Final Brix of retentate: 9 (pH 6.91 at 25.0° C. after cooling with plate heat exchanger)
Collected approximately 11 L without dilution and 6 L with some dilution

### Hydrolysis and Cholesterol Sequestration Method

The MF retentate (Table 4.1) was heated to 50° C. and adjusted to pH 9.31. Novozyme Alcalase 2.4 L FG (30 mL) and Wacker β-cyclodextrin (50 g) were added directly to the retentate tank. The mixture was incubated for 120 min in the MF plant, which continued to operate throughout to provide mixing. The target conditions were pH 9.0 and 50° C. Sodium hydroxide (470 g, 4M) was added to maintain the pH. The relevant details recorded are presented in Table 4.3

TABLE 4.3
Details AMF serum retentate hydrolysis.

| | | | pH | | NaOH |
|---|---|---|---|---|---|
| | | Temp | Pre | Post | added |
| Time | Comment | (° C.) | NaOH | NaOH | (g) |
| 1304 | Commence heating | 15.3 | 6.85 | — | — |
| 1309 | Added (too much) hot | 27.0 | 6.76 | — | — |
| | water | | | | |
| 1337 | Adjust pH (with NaOH/ | 51.8 | 6.64 | 9.31 | 140 |
| | HCl) | | | | |
| 1342 | Add Alcalase, Cyclodextrin | 50.0 | 9.31 | — | — |
| 1351-4 | Adjust pH | 51.8 | 7.03 | 9 | 180 |
| 1355 | Monitor temp and pH | 50.5 | 8.57 | 8.88 | 50 |
| 1404 | Monitor temp and pH | 51.2 | 8.22 | 9.10 | 90 |
| 1420 | Monitor temp and pH | 48.4 | 8.46 | 9.15 | 80 |
| 1449 | Monitor temp and pH | 49.4 | 8.44 | 9.24 | 90 |
| 1535 | Monitor temp and pH | 49.0 | 8.62 | 9.13 | 70 |
| 1603 | Commence MF | 47.5 | 8.97 | — | — |

### Results

A phospholipid enriched product was manufactured, but the product was not cholesterol free. This outcome does not exclude the possibility of cholesterol sequestration and removal by cyclodextrin.

TABLE 4.4
Composition of powders derived from AMF serum by
MF and hydrolysis.

| | | | AMF | AMF |
|---|---|---|---|---|
| | | | serum | serum |
| | AMF | AMF | MF | MF |
| | serum | serum | RET/HYD/ | RET/HYD/ |
| COMPONENT | MF PERM | MF RET | PERM | RET |
| Ash (%) | 8.4 | 6.2 | 8.8 | 8.9 |
| Moisture (%) | 5.3 | 5.5 | 7.4 | 2.1 |
| Fat (%) | 0.25 | 24.7 | 0.4 | 62 |
| Phospholipids | <0.1 | 23.1 | <0.1 | 52.5 |
| (%) | | | | |
| Non-PL fat (%) | 0.25 | 1.6 | 0.4 | 9.5 |
| Cholesterol | <1 | 290 | <1 | 670 |
| (mg/100 g) | | | | |
| Lactose (%) | 79.6 | 4.3 | 5.8 | 1.4 |
| Protein (%) | 5.4 | 57.9 | 74.2 | 27.3 |
| Phospholipids | 0.0 | 93.5 | 0.0 | 84.7 |
| (% total fat) | | | | |
| Phospholipids | | | | 48.6 |
| (NMR, %) | | | | |
| PC (% PL) | | | | 26.6 |
| PI (% PL) | | | | 8.9 |
| PS (% PL) | | | | 11.6 |
| 2LPC (% PL) | | | | 1.0 |
| PE (% PL) | | | | 28.4 |
| SM (% PL) | | | | 18.7 |
| DHSM (% PL) | | | | 3.2 |
| 2LPE (% PL) | | | | 1.1 |
| Total SM* | | | | 21.9 |
| (% PL) | | | | |

### Example 5

### Process for the Preparation of a Phospholipid Enriched Fraction from AMF Serum Derivatives Using Alcalase to Prepare CRD29NOV06G1

### Raw Materials

The AMF serum (54.57 kg) used were collected from Rochester in 15 L drums and transported to Cobram.

### Membrane Filtration Methodology

The membrane filtration plant (Model 92 Laboratory Unit, Filtration Engineering Co. Inc.) was fitted with Desal JX (0.3 μm MF) membranes and a plate cooler was fitted to the valve 5 outlet to allow heating/cooling of the retentate. All MF was undertaken at 0 Bar (no valve 2 closure, valve 1 and 4 remained closed) and collection of retentate commenced only after permeate composition had equilibrated. The recorded data for pre- (Table 5.1) and post-hydrolysis (Table 5.2) is presented below.

TABLE 5.1
Details of the MF concentration and diafiltration
of AMF serum.

| | Permeate | | |
|---|---|---|---|
| | removed | DF water | |
| Starting mass (kg) | (kg) | added (L) | Temp (° C.) |
| 54.57 + system volume | 44 | trickle 0 | 14.8 at start, |
| | 48 | | MF'd at 47-50 |

TABLE 5.2
Details of the MF concentration and
2 × diafiltration of AMF serum hydrolysate.

| Starting | Permeate removed | DF water | |
|---|---|---|---|
| mass (kg) | (kg) | added (L) | Temp (° C.) |
| 40 | 28.35 | Unknown | 50 |
| | 55 (0.0-0.2 brix) | trickle 0 | 54.5 |
| | | | (pH7.68) |

### Hydrolysis Method

The MF retentate (Table 5.1) was held at 50° C. and adjusted to pH 9.13. Novozyme Alcalase 2.4L FG (32 mL) was added directly to the retentate tank. The mixture was incubated for 90 min in the MF plant, which continued to operate throughout to provide mixing. The target conditions were pH 9.0 and 50° C. Sodium hydroxide (400 g, 2M) was added to maintain the pH. The relevant details recorded are presented in Table 5.3.

TABLE 5.3
Details AMF serum retentate hydrolysis.

| | | | pH | | NaOH |
|---|---|---|---|---|---|
| | | Temp | Pre | Post | added |
| Time | Comment | (° C.) | NaOH | NaOH | (g) |
| | Start | 50 | 6.51 | 9.13 | 120 |
| 1400 | Added 32 mL Alcalase | | 9.07 | — | — |
| 1405 | Adjust pH | | 7.00 | >9 | 170 |
| 1435 | Adjust pH | | 7.20 | >9 | 230 |
| 1525 | Monitor pH, commence MF | | 8.0 | — | — |

### Batch Pasteurisation Method

The MF Ret/Hyd/Ret was removed from the MF and placed in a 25 L boiler. The boiler was placed in hot water and stirred. The maximum temperature reached was 70.5° C. and this temperature was held for 30 s. The material was cooled in the freezer and then freeze-dried for 48 h at 43° C.

### Results & Discussion

The composition of the product CRD29NOV06G1 is presented Table 5.5. The results show that CRD29NOV06G1 did reach the required phospholipid content on a mass basis, but did not reach the required phospholipid content on a fat basis (aim at least 80%, actual 64.4). Raw material variation is a probable cause of this difference. It appears that the total fat content of the starting material was higher than normal.

TABLE 5.5
Composition of CRD29NOV06G1 powder derived from
AMF serum by MF and hydrolysis.

| | AMF serum MF |
|---|---|
| COMPONENT | RET/HYD/RET |
| Ash (%) | 7.1 |
| Moisture (%) | 1.7 |
| Fat (%) | 72.1 |
| Phospholipids (%) | 46.4 |
| Non-PL fat (%) | 25.7 |
| Cholesterol | 590 |
| (mg/100 g) | |
| Lactose (%) | 1.3 |
| Protein (%) | 19.8 |
| Phospholipids | 64.4 |
| (% total fat) | |
| SPC (CFU/g) | 720 |
| Coliforms (CFU/g) | ND |
| Thermophiles (CFU/g) | 200 |
| Phospholipids (NMR, %) | 40.5 |
| PC (% PL) | 26.6 |
| PI (% PL) | 8.3 |
| PS (% PL) | 12.0 |
| 2LPC (% PL) | 0.6 |
| PE (% PL) | 29.3 |
| SM (% PL) | 18.9 |
| DHSM (% PL) | 3.7 |
| 2LPE (% PL) | 0.6 |
| Total SM* | 22.6 |
| (% PL) | |
| Fatty | |
| acid profile (% | |
| fatty acids) | |
| C: 4.0 | 3.5 |
| C: 6.0 | 1.5 |
| C: 8.0 | 0.8 |
| C: 10.0 | 2 |
| C: 10.1 ω1 cis | 0.1 |
| C: 12.0 | 2.5 |
| C: 12.1 ω9 cis | <0.1 |
| C: 12.1 ω6 cis | <0.1 |
| C: 12.1 ω3 cis | <0.1 |
| C: 14.0 iso | 0.1 |
| C: 14.0 | 9.1 |
| C: 14.1 ω5 cis | 0.9 |
| C: 15.0 ante-iso | 0.5 |
| C: 15.0 | 1 |
| C: 16.0 iso | 0.3 |
| C: 15.1 ω5 cis | <0.1 |
| C: 16.0 | 26.8 |
| C: 16.1 trans | <0.1 |
| C: 16.1 ω7 cis | 1.6 |
| C: 17.0 iso | 0.5 |
| C: 17.0 ante-iso | 0.7 |
| C: 17.0 | 0.7 |
| C: 17.1 ω7 cis | 0.3 |
| C: 18.0 iso | <0.1 |
| C: 18.0 | 11 |
| C: 18.1 trans | 2.4 |
| C 18.1 cis | 23.9 |
| C: 18.2 trans | 0.5 |
| C: 18.2 cis9 trans12 | 0.3 |
| C: 18.2 trans9 cis12 | <0.1 |
| C: 18.2 ω6 cis | 3.7 |
| C: 18.3 trans | <0.1 |
| C: 18.3 ω6 cis | <0.1 |
| C: 18.3 ω3 cis | 1 |
| C: 20.0 | 0.1 |
| C: 18.2 conj | 1.2 |
| C: 18.4 ω3 | <0.1 |
| C: 20.1 ω9 cis | 0.1 |
| C: 20.2 ω6 cis | <0.1 |
| C: 20.3 ω6 cis | 0.4 |
| C: 20.4 ω6 cis | <0.1 |
| C: 20.3 ω3 cis | 0.4 |
| C: 22.0 | 0.2 |
| C: 20.4 ω3 | <0.1 |
| C: 22.1 ω9 cis | <0.1 |
| C: 20.5 ω3 cis | <0.1 |
| C: 22.2 ω6 cis | 0.1 |
| C: 21.5 ω3 | <0.1 |
| C: 22.5 ω6 | <0.1 |
| C: 24.0 | <0.1 |
| C: 24.1 ω9 cis | <0.1 |
| C: 22.5 ω3 | <0.1 |
| C: 22.6 ω3 cis | <0.1 |
| Others | 1.7 |

### Example 6

### Process for the Preparation of a Phospholipid Enriched Fraction from AMF Serum Derivatives Using Alcalase to Prepare CRD5JAN07G1

### Raw Materials

AMF serum (6 drums) was collected from Rochester in 15 L drums, transported to Cobram and then stored cool overnight. AMF serum was produced in a run processing mainly whey cream, rather than choice cream.

### Membrane Filtration Methodology

The membrane filtration plant (Model 92 Laboratory Unit, Filtration Engineering Co. Inc.) was fitted with Desal JX (0.3 μm MF) membranes and a plate cooler was fitted to the valve 5 outlet to allow heating/cooling of the retentate. All MF was undertaken at 2 Bar (limited valve 2 closure, valve 1 and 4 remained closed) and collection of retentate commenced only after permeate composition had equilibrated. The recorded data for pre- (Table 6.1) and post-hydrolysis (Table 6.2) is presented below.

TABLE 6.1
Details of the MF concentration and diafiltration
of AMF serum.

| | | DF | |
|---|---|---|---|
| | | water | |
| | Permeate | added | |
| Starting mass (kg) | removed (kg) | (L) | Temp (° C.) |
| 69.45 + system volume | 51 (4.5 brix) | trickle 0 | 12.8 at |
| | 84 (0.8 brix) | | start, |
| | | | target 10 |

End 12brix, pH7.08

TABLE 6.2
Details of the MF concentration and
2 × diafiltration of AMF serum hydrolysate.

| Starting | | DF water | |
|---|---|---|---|
| mass | | added | |
| (kg) | Permeate removed (kg) | (L) | Temp (° C.) |
| 50 | 38 (1.5 brix) | Unknown | 50 |
| | 46 (0.0 brix, pH7.08) | trickle 0 | 54.5 (pH7.68) |

End 12 kg, pH7.06, pH7.06

### Hydrolysis Method

The MF retentate (Table 6.1) was held at 50° C. and adjusted to pH 9.06. Novozyme Alcalase 2.4L FG (30 mL) was added directly to the retentate tank. The mixture was incubated for 90 min in the MF plant, which continued to operate throughout to provide mixing. The target conditions were pH 9.0 and 50° C. Sodium hydroxide (560 g, 2M) was added to maintain the pH. The relevant details recorded are presented in Table 6.3.

TABLE 6.3
Details AMF serum retentate hydrolysis.

| | | | pH | | NaOH |
|---|---|---|---|---|---|
| | | Temp | Pre | Post | added |
| Time | Comment | (° C.) | NaOH | NaOH | (g) |
| 200 | Start | 45.6 | 7.08 | 9.06 | 190 |
| 205 | Add 30 mL Alcalase | 52 | 6.97 | 9.2 | 370 |
| 213 | Monitor | 53.7 | 8.08 | — | — |
| 240 | Monitor | 48.8 | 7.49 | — | — |
| 310 | Monitor | 50.0 | 7.20 | — | — |
| 324 | Monitor pH, commence MF | 49.9 | 8.0 | — | — |

### Batch Pasteurisation and Drying Methods

The MF Ret/Hyd/Ret was removed from the MF and placed in a 25 L boiler. The boiler was placed in hot water and stirred. The maximum temperature reached was 70.5° C. and this temperature was held for 30 s. The material was cooled in the freezer and then freeze-dried for 48 h at 43° C.

### Results & Discussion

The composition of the product CRD5JAN07G1 is presented Table 6.4. The results show that CRD5JAN07G1 reached the required phospholipid content on both a mass basis and fat basis. Table 6.5 compares the fatty acid profile of the materials produced during EXAMPLES 5 and 6.

TABLE 6.4
Composition of powders derived from AMF serum by
MF and hydrolysis.

| | | | | AMF | |
|---|---|---|---|---|---|
| | | AMF | AMF | serum | AMF |
| | | serum | serum | MF | serum MF |
| | AMF | MF | MF | RET/HYD/ | RET/HYD/ |
| COMPONENT | serum | PERM | RET | PERM | RET |
| Ash (%) | 6.8 | 9.1 | 4.5 | 6.5 | 5.9 |
| Moisture (%) | 2.2 | 2.4 | 1.9 | 3 | 1.1 |
| Fat (%) | 14.2 | <0.1 | 32 | 0.95 | 57.1 |
| Phospholipids | 10.2 | 0.4 | 15 | <0.1 | 47.9 |
| (%) | | | | | |
| Non-PL fat (%) | 4 | 0 | 17 | 0.95 | 9.2 |
| Cholesterol | 130 | <1 | 320 | <1 | 610 |
| (mg/100 g) | | | | | |
| Lactose (%) | 47.9 | 84.6 | 1 | 1.8 | 0.7 |
| Protein (%) | 26.7 | 4.48 | 56.6 | 85.4 | 27.6 |
| Phospholipids | 71.8 | — | 46.9 | <10 | 83.9 |
| (% total fat) | | | | | |
| Phospholipids | | | | | 40.8 |
| (NMR, %) | | | | | |
| PC (% PL) | | | | | 26.2 |
| PI (% PL) | | | | | 8.8 |
| PS (% PL) | | | | | 12.5 |
| 2LPC (% PL) | | | | | 0.7 |
| PE (% PL) | | | | | 29.2 |
| SM (% PL) | | | | | 19.1 |
| DHSM (% PL) | | | | | 3.1 |
| 2LPE (% PL) | | | | | 0.5 |
| Total SM | | | | | 22.2 |
| (% PL) | | | | | |

TABLE 6.5
A comparison of the fatty acid profile from the
MF RET/HYD/MF RET obtained in EXAMPLES 5 and 6.

| | EX. 5 | EX. 6 |
|---|---|---|
| C: 4.0 | 3.5 | 2.6 |
| C: 6.0 | 1.5 | 1.4 |
| C: 8.0 | 0.8 | 0.7 |
| C: 10.0 | 2 | 1.9 |
| C: 10.1 ω1 cis | 0.1 | 0.2 |
| C: 12.0 | 2.5 | 2.5 |
| C: 12.1 ω9 cis | <0.1 | 0.1 |
| C 12.1 ω6 cis | <0.1 | 0.1 |
| C: 12.1 ω3 cis | <0.1 | 0.1 |
| C: 14.0 iso | 0.1 | 0.1 |
| C: 14.0 | 9.1 | 9 |
| C: 14.1 ω5 cis | 0.9 | 0.9 |
| C: 15.0 ante-iso | 0.5 | 0.5 |
| C: 15.0 | 1 | 1 |
| C: 16.0 | 26.8 | 28.9 |
| C: 15.1 ω5 cis | <0.1 | <0.1 |
| C: 16.0 iso | 0.3 | 0.2 |
| C: 16.1 trans | <0.1 | 0.6 |
| C: 16.1 ω7 cis | 1.6 | 1.6 |
| C: 17.0 iso | 0.5 | 0.3 |
| C: 17.0 ante-iso | 0.7 | 0.6 |
| C: 17.0 | 0.7 | 0.6 |
| C: 17.1 ω7 cis | 0.3 | 0.3 |
| C: 18.0 iso | <0.1 | 0.1 |
| C: 18.0 | 11 | 10.2 |
| C 18.1 cis | 23.9 | 24 |
| C: 18.1 trans | 2.4 | 1.8 |
| C: 18.2 cis9 tr12 | 0.3 | <0.1 |
| C: 18.2 conj | 1.2 | 0.9 |
| C: 18.2 tr9 cis12 | <0.1 | <0.1 |
| C: 18.2 trans | 0.5 | 0.2 |
| C: 18.2 ω6 cis | 3.7 | 3.6 |
| C: 18.3 trans | <0.1 | 0.2 |
| C: 18.3 ω3 cis | 1 | 0.8 |
| C: 18.3 ω6 cis | <0.1 | <0.1 |
| C: 18.4 ω3 | <0.1 | <0.1 |
| C: 20.0 | 0.1 | 0.1 |
| C: 20.1 ω9 cis | 0.1 | 0.2 |
| C: 20.2 ω6 cis | <0.1 | 0.1 |
| C: 20.3 ω3 cis | 0.4 | 0.4 |
| C: 20.3 ω6 cis | 0.4 | 0.4 |
| C: 20.4 ω3 | <0.1 | <0.1 |
| C: 20.4 ω6 cis | <0.1 | <0.1 |
| C: 20.5 ω3 cis | <0.1 | 0.2 |
| C: 21.5 ω3 | <0.1 | <0.1 |
| C: 22.0 | 0.2 | 0.2 |
| C: 22.1 ω9 cis | <0.1 | <0.1 |
| C: 22.2 ω6 cis | 0.1 | 0.1 |
| C: 22.5 ω3 | <0.1 | 0.4 |
| C: 22.5 ω6 | <0.1 | <0.1 |
| C: 22.6 ω3 cis | <0.1 | <0.1 |
| C: 24.0 | <0.1 | <0.1 |
| C: 24.1 ω9 cis | <0.1 | <0.1 |
| Others | 1.7 | 1.9 |

### Example 7

### Production of CRD14JUN07G1

### Raw Materials

AMF serum (214 kg) was collected from Rochester in drums, transported to Cobram and then stored cool overnight. The AMF serum was probably derived from 50% whey cream and 50% choice cream.

### Membrane Filtration Methodology

The membrane filtration plant (Model 92 Laboratory Unit, Filtration Engineering Co. Inc.) was fitted with Desal JX (0.3 μm MF) membranes and a plate heat exchanger (PHE) was fitted to the valve 5 outlet to allow heating/cooling of the retentate. All MF was undertaken at 0 Bar (no valve 2, 3 or 5 closure; valve 1 and 4 remained fully closed) and collection of retentate commenced only after permeate composition had equilibrated.

### Hydrolysis Method

The MF retentate was held at 50° C. and adjusted to approximately pH 9.0. Novozyme Alcalase 2.4L FG (60 mL) was added directly to the retentate tank. The mixture was incubated for 90 min in the MF plant, which continued to operate throughout to provide mixing. The target conditions were pH 9.0 and 50° C. Sodium hydroxide (1.8 kg, 2M) was added to maintain the pH. The relevant details recorded are presented in Table 7.1.

### Batch Pasteurisation and Drying Methods

The MF Ret/Hyd/Ret was removed from the MF and placed in a 25 L boiler. The boiler was placed in 80° C. hot water and stirred continually. The maximum temperature reached was 71° C. and pasteurization was deemed to have occurred after 3 min at greater than 69° C. The material was cooled to 25° C. by placing the boiler in chilled water and then freeze-dried for 72 h at 40° C.

### Storage

The dried MF Ret/Hyd/Ret was vacuum-packed into sealed plastic bags, then over-bagged in sealed light-proof foil pouches and stored at −40° C. The total yield was 2848 g.

TABLE 7.1
Details AMP serum retentate hydrolysis.

| | Solids | | | RET pH | | Cumulative | PERM | |
|---|---|---|---|---|---|---|---|---|
| | (Brix) | | Temp | Pre | Post | NaOH added | removed | Estimate |
| Time | RET | PERM | (° C.) | NaOH | NaOH | (kg) | (kg) | RET (kg) |

Wednesday, 13^{th} June.

| 1045 | Commence process by adding 134.3 kg AMF serum. Start MF. | | | | |
|---|---|---|---|---|---|
| 1059 | | | 11.2 | 0 | |
| 1142 | | | 13.1 | | |
| 1234 | | | 14.2 | | |
| 1240 | Add remaining 79.9 kg AMF serum (total 214 kg). | | | | |
| 1331 | | | 16.4 | | |
| 1402 | 13 | 5.5 | 17.8 | 140 | 74 |
| 1434 | 15 | — | 19.2 | 157 | 57 |
| 1443 | | | 19.8 | | |
| 1443 | Commence cooling to allow hot water addition during DF. | | | | |
| 1452 | 16 | — | 8.4 | 166 | 48 |
| 1455 | Drum half full. Commence DF water addition and fill drum by | | | | |
| | 1459. Commence DF. | | | | |
| 1515 | — | 2.75 | 18.9 | | |
| 1553 | 10.5 | 2.5 | 13.4 | 41 | |
| 1647 | 14.0 | — | 11.6 | 66.5 | |
| 1739 | 17.2 | 2.75 | 16.3 | 89.1 | |

| | |
|---|---|
| 1740 | Cool MF retentate prior to collection and storage in cool |
| | room. |
| | Mass collected was 41.5 kg. |

Thursday, 14^{th} June.

| 1047 | Added 39.99 kg MF retentate to membrane plant. | | | | | | |
|---|---|---|---|---|---|---|---|
| 1047 | 16 | — | 8.8 | 6.83 | — | — | |
| 1051 | Add approximately 80L (filled drum) hot water and apply | | | | | | |
| | heat to PHE. | | | | | | |
| 1113 | Add 2M NaOH to raise mixture to the starting pH. | | | | | | |
| 1113 | | | 47.0 | 6.83 | 9.27 | 0.58 | |
| 1114 | Add 60 mL Alcalase. Start hydrolysis. | | | | | | |
| 1117 | | | 47.0 | 8.00 | 9.02 | 0.32 | |
| 1120 | Water flowing through PHE approximately 57° C. at this point. | | | | | | |
| 1125 | | | 47.9 | 8.00 | 9.05 | 0.36 | |
| 1136 | | | 50.0 | 8.13 | 9.05 | 0.34 | |
| 1203 | | | 47.3 | 8.08 | 9.21 | 0.47 | |
| 1229 | | | 46.7 | 8.66 | 9.24 | 0.30 | |
| 1235 | | | 48.3 | 9.17 | | | |
| 1235 | Commence permeate removal. | | | | | | |
| 1240 | Sampled MF ret/hyd/permeate (2L). | | | | | | |
| 1240 | 5.5 | 2.75 | | | | | 17 |
| 1312 | 8.0 | 2.8 | 50.9 | 8.54 | | | 67 |
| 1342 | 13 | — | 45.2 | 8.34 | | | 99 |
| 1343 | Add DF water (hot) to fill drum. | | | | | | |
| 1345 | 2.5 | 0 | 58 | 8.22 | | | 0 |
| 1350 | Add 0.2 kg 2M HCl to adjust pH. Left PHE on too long so | | | | | | |
| | temperature peaked at 58° C. | | | | | | |
| 1350 | | | 57.7 | 7.45 | | | 13 |
| 1417 | 4.5 | 0 | 52.7 | 7.35 | | | 44 |
| 1443 | | | | | | | 76 |
| 1515 | 17 | | 41.4 | 7.31 | | | 96.5 |

| | |
|---|---|
| 1517 | Recover final phospholipid enriched material (18 kg, 16 |
| | brix). |

### Results & Discussion

A phospholipid enriched material with suitable properties was produced in sufficient amounts for further work (Table 7.4).

TABLE 7.4
Composition of powders derived from AMF serum by
MF and hydrolysis.

| | | | | AMF | AMF |
|---|---|---|---|---|---|
| | | AMF | AMF | serum | serum |
| | | serum | serum | MF | MF |
| | AMF | MF | MF | RET/HYD/ | RET/HYD/ |
| Component | serum | PERM | RET | PERM | RET |
| Ash (%) | 6.8 | 8.7 | 5.3 | 8.3 | 8.8 |
| Moisture (%) | 4.7 | 3.1 | 1.2 | 3.5 | 1.4 |
| Fat (%) | 11.3 | 0.7 | 25.9 | 0.3 | 57.6 |
| Phospholipids | 13.6 | 0.2 | 28.3 | <0.1 | 56.9 |
| (%) | | | | | |
| Non-PL fat (%) | −2.3 | 0.5 | −2.4 | 0.3 | 0.7 |
| Cholesterol | 160 | <1 | 340 | <1 | 740 |
| (mg/100 g) | | | | | |
| Lactose (%) | 46.7 | 82.9 | 7.4 | 15.2 | 1.6 |
| Protein (%) | 28.9 | 6.1 | 56.3 | 70.8 | 26.8 |
| Phospholipids | 100 | 29 | 100 | 0 | 98.8 |
| (% total fat) | | | | | |
| Phospholipids | | | | | 49.8 |
| (NMR, %) | | | | | |
| PC (% PL) | | | | | 26.2 |
| PI (% PL) | | | | | 8.6 |
| PS (% PL) | | | | | 12.1 |
| 2LPC (% PL) | | | | | 0.7 |
| PE (% PL) | | | | | 29.6 |
| SM (% PL) | | | | | 18.9 |
| DHSM (% PL) | | | | | 3.3 |
| 2LPE (% PL) | | | | | 0.7 |
| Total SM | | | | | 22.2 |
| (% PL) | | | | | |
| Microbes | | | | | |
| (cfu/g) | | | | | |
| Coliforms | | | | | ABSENT |
| SPC | | | | | 1000 |
| Yeasts | | | | | <10 |
| Moulds | | | | | <10 |
| Salmonella | | | | | ABSENT |
| Bacillus cereus | | | | | <100 |
| Thermophiles | | | | | 500 |
| Coliforms | | | | | ABSENT |

TABLE 5
A comparison of the fatty acid profile from the MF
RET/HYD/MF RET obtained in EXAMPLES 5, 6 and 7.

| Fatty acid | TRIAL 5 | TRIAL 6 | TRIAL 7 |
|---|---|---|---|
| 4:0 | 3.5 | 2.6 | 1.7 |
| 6:0 | 1.5 | 1.4 | 1 |
| 8:0 | 0.8 | 0.7 | 0.6 |
| 10:0 | 2 | 1.9 | 1.4 |
| 10:1 ω1 cis | 0.1 | 0.2 | 0.1 |
| 12:0 | 2.5 | 2.5 | 2.1 |
| 12:1 ω9 cis | <0.1 | 0.1 | <0.1 |
| 12:1 ω6 cis | <0.1 | 0.1 | <0.1 |
| 12:1 ω3 cis | <0.1 | 0.1 | <0.1 |
| 14:0 iso | 0.1 | 0.1 | 0.1 |
| 14:0 | 9.1 | 9 | 7.1 |
| 14:1 ω5 cis | 0.9 | 0.9 | 0.7 |
| 15:0 ante-iso | 0.5 | 0.5 | 0.3 |
| 15:0 | 1 | 1 | 0.9 |
| 16:0 | 26.8 | 28.9 | 23.3 |
| 15:1 ω5 cis | <0.1 | <0.1 | <0.1 |
| 16:0 iso | 0.3 | 0.2 | 0.2 |
| 16:1 trans | <0.1 | 0.6 | 0.1 |
| 16:1 ω7 cis | 1.6 | 1.6 | 1.5 |
| 17:0 iso | 0.5 | 0.3 | 0.3 |
| 17:0 ante-iso | 0.7 | 0.6 | 0.5 |
| 17:0 | 0.7 | 0.6 | 0.5 |
| 17:1 ω7 cis | 0.3 | 0.3 | 0.3 |
| 18:0 iso | <0.1 | 0.1 | 0.1 |
| 18:0 | 11 | 10.2 | 11.8 |
| 18:1 cis | 23.9 | 24 | 29.2 |
| 18:1 trans | 2.4 | 1.8 | 2.7 |
| 18:2 cis9 tr12 | 0.3 | <0.1 | 0.6 |
| 18:2 conj | 1.2 | 0.9 | 1.2 |
| 18:2 tr9 cis12 | <0.1 | <0.1 | 0.2 |
| 18:2 trans | 0.5 | 0.2 | 0.4 |
| 18:2 ω6 cis | 3.7 | 3.6 | 5.1 |
| 18:3 trans | <0.1 | 0.2 | <0.1 |
| 18:3 ω3 cis | 1 | 0.8 | 1.1 |
| 18:3 ω6 cis | <0.1 | <0.1 | <0.1 |
| 18:4 ω3 | <0.1 | <0.1 | <0.1 |
| 20:0 | 0.1 | 0.1 | 0.2 |
| 20:1 ω9 cis | 0.1 | 0.2 | 0.1 |
| 20:2 ω6 cis | <0.1 | 0.1 | <0.1 |
| 20:3 ω3 cis | 0.4 | 0.4 | <0.1 |
| 20:3 ω6 cis | 0.4 | 0.4 | 0.5 |
| 20:4 ω3 | <0.1 | <0.1 | <0.1 |
| 20:4 ω6 cis | <0.1 | <0.1 | 0.7 |
| 20:5 ω3 cis | <0.1 | 0.2 | 0.3 |
| 21:5 ω3 | <0.1 | <0.1 | <0.1 |
| 22:0 | 0.2 | 0.2 | 0.2 |
| 22:1 ω9 cis | <0.1 | <0.1 | <0.1 |
| 22:2 ω6 cis | 0.1 | 0.1 | <0.1 |
| 22:5 ω3 | <0.1 | 0.4 | 0.8 |
| 22:5 ω6 | <0.1 | <0.1 | <0.1 |
| 22:6 ω3 cis | <0.1 | <0.1 | <0.1 |
| 24:0 | <0.1 | <0.1 | 0.1 |
| 24.1 ω9 cis | <0.1 | <0.1 | <0.1 |
| Others | 1.7 | 1.9 | 2 |

### Example 8

### Ethanol Fractionation of CRD14JUN07G1 to Produce CRD4JUL07G1 and CRD4JUL07G2

### Raw Materials

The phospholipid enriched fraction prepared in Example 7 (1295 g) (CRD14JUN07G1) was removed from frozen storage (−40° C.) and defrosted by immersion in absolute ethanol.

### Ethanol Extraction Methodology

The fraction was initially processed in approximately 250 g batches. Each 250 g batch was added to 400 mL ethanol in a 1 L beaker and stirred for several minutes. The slurry was filtered through a Whatman #113 filter disk placed in a Buchner funnel sitting a vacuum flask. A vacuum was applied to speed the filtration process. After the five batches were processed, the sediment cakes were pooled, resuspended in 2 L ethanol and the filtered as described above. The ethanol filtrate from both extractions was pooled and clarified by filtration through identical apparatus as described above, except a Whatman #1 disk was used. The ethanol extract eventually collected totalled 5.25 L.

### Ethanol-Soluble Fraction

Ethanol was removed from the ethanol-soluble fraction (ESF) by means of a Buchi R-114 rotary evaporator over a period of seven hours. The vacuum was applied by a Barnant Company Pressure Station and the temperature of the water bath was approximately 63-65° C. A 1 L evaporating flask was three-quarters filled with ESF and then rotary evaporated to approximately one-quarter full. The remaining ESF was removed and stored until latter. The remaining 2 L ESF was then reduced to approximately 900 mL in two batches and then gradually rotary evaporated until little further ethanol appeared to be entering the condensate collection flask. The remaining ESF was stored overnight at −20° C.

### Ethanol-Insoluble Fraction

The ethanol-insoluble fraction (EIF) was resuspended in 3 L absolute ethanol and stirred for a prolonged period (7 h). The insoluble material was collected by filtration through a Whatman #113 filter disk and then rinsed with 200 mL ethanol while the vacuum remained. The insoluble material was stored overnight at −4° C. and then some ethanol was removed in a freeze-dryer. The remaining ethanol was removed by air drying the ethanol insoluble material overnight in a fume-hood.

### Yield, Storage and Transport

The yields were 425 g ESF and 750 g EIF. The ESF (60 g each) was placed in a plastic 120 mL container prior to storage process continuing as now described. Both the ESF and EIF (150 g each) portions were vacuum-packed in a plastic bag, over-bagged in a foil pouch and then stored −40° C.

### Results & Discussion

TABLE 8.1
Composition of powders derived from AMF serum by
MF and hydrolysis.

| | | ESF | EIF |
|---|---|---|---|
| | AMF serum MF | (ethanol- | (ethanol- |
| | RET/HYD/RET | soluble | insoluble |
| Component | (PLRME) | fraction) | fraction) |
| Ash (%) | 8.8 | 3 | 10.6 |
| Moisture (%) | 1.4 | 8.5 | 6.7 |
| Fat (%) | 57.6 | 76.3 | 35.4 |
| Phospholipids (%) | 56.9 | 68 | 38.6 |
| Non-PL fat (%) | 0.7 | 8.3 | −12.6 |
| Cholesterol | 740 | 1600 | 84 |
| (mg/100 g) | | | |
| Lactose (%) | 1.6 | 0.2 | 2.1 |
| Protein (%) | 26.8 | 9.4 | 37 |
| Phospholipids | 98.8 | 89 | 109 |
| (% total fat) | | | |

TABLE 8.2
A comparison of the fatty acid profiles of the
phospholipid enriched fraction and ESF and EIF.

| | | ESF | EIF |
|---|---|---|---|
| | AMF serum MF | (ethanol- | (ethanol- |
| | RET/HYD/RET | soluble | insoluble |
| Fatty acid | (PLRME) | fraction) | fraction) |
| 4:0 | 1.7 | 2.1 | 0.2 |
| 6:0 | 1 | 1.3 | 0.1 |
| 8:0 | 0.6 | 0.7 | 0.1 |
| 10:0 | 1.4 | 1.6 | 0.3 |
| 10:1 ω1 cis | 0.1 | 0.2 | 0.1 |
| 12:0 | 2.1 | 2.5 | 0.7 |
| 12:1 ω9 cis | <0.1 | 0.1 | <0.1 |
| 12:1 ω6 cis | <0.1 | <0.1 | <0.1 |
| 12:1 ω3 cis | <0.1 | 0.1 | <0.1 |
| 14:0 iso | 0.1 | 0.1 | 0.1 |
| 14:0 | 7.1 | 8.2 | 3 |
| 14:1 ω5 cis | 0.7 | 0.7 | 0.1 |
| 15:0 ante-iso | 0.3 | 0.4 | 0.2 |
| 15:0 | 0.9 | 1 | 0.4 |
| 16:0 | 23.3 | 26.1 | 14.1 |
| 15:1 ω5 cis | <0.1 | 0.2 | 0.1 |
| 16:0 iso | 0.2 | 0.2 | 0.3 |
| 16:1 trans | 0.1 | 0.3 | 0.2 |
| 16:1 ω7 cis | 1.5 | 1.6 | 1.3 |
| 17:0 iso | 0.3 | 0.3 | 0.2 |
| 17:0 ante-iso | 0.5 | 0.6 | 0.4 |
| 17:0 | 0.5 | 0.5 | 0.5 |
| 17:1 ω7 cis | 0.3 | 0.3 | 0.3 |
| 18:0 iso | 0.1 | 0.1 | 0.1 |
| 18:0 | 11.8 | 6.9 | 16.3 |
| 18:1 cis | 29.2 | 24 | 35.8 |
| 18:1 trans | 2.7 | 1.9 | 2.6 |
| 18:2 cis9 tr12 | 0.6 | 0.5 | 0.7 |
| 18:2 conj | 1.2 | 0.8 | 1.4 |
| 18:2 tr9 cis12 | 0.2 | 0.1 | 0.1 |
| 18:2 trans | 0.4 | 0.3 | 0.4 |
| 18:2 ω6 cis | 5.1 | 4.2 | 8.1 |
| 18:3 trans | <0.1 | 0.2 | 0.3 |
| 18:3 ω3 cis | 1.1 | 1.1 | 1.4 |
| 18:3 ω6 cis | <0.1 | <0.1 | <0.1 |
| 18:4 ω3 | <0.1 | <0.1 | <0.1 |
| 20:0 | 0.2 | 0.2 | 0.3 |
| 20:1 ω9 cis | 0.1 | <0.1 | 0.1 |
| 20:2 ω6 cis | <0.1 | <0.1 | <0.1 |
| 20:3 ω3 cis | <0.1 | <0.1 | <0.1 |
| 20:3 ω6 cis | 0.5 | 0.3 | 1.3 |
| 20:4 ω3 | <0.1 | <0.1 | <0.1 |
| 20:4 ω6 cis | 0.7 | 0.4 | 1.1 |
| 20:5 ω3 cis | 0.3 | 0.2 | 0.5 |
| 21:5 ω3 | <0.1 | <0.1 | <0.1 |
| 22:0 | 0.2 | 2 | 0.9 |
| 22:1 ω9 cis | <0.1 | <0.1 | 0.3 |
| 22:2 ω6 cis | <0.1 | 2.4 | 0.8 |
| 22:5 ω3 | 0.8 | 0.4 | 1.4 |
| 22:5 ω6 | <0.1 | <0.1 | <0.1 |
| 22:6 ω3 cis | <0.1 | <0.1 | <0.1 |
| 24:0 | 0.1 | 2 | 0.9 |
| 24.1 ω9 cis | <0.1 | 0.4 | 0.1 |
| Others | 2 | 2.5 | 2.4 |

### Example 9

### Production of CRD17JUN07G1 with the Mammalian (Non-Fungal, Non-Bacterial) Protease Trypsin

### Method

### Raw Materials

AMF serum (45 L) was collected from Rochester in drums and then frozen until further processing.

### Membrane Filtration Methodology

The membrane filtration plant (Model 92 Laboratory Unit, Filtration Engineering Co. Inc.) was fitted with Desal JX (0.3 μm MF) membranes and a plate heat exchanger (PHE) was fitted to the valve 5 outlet to allow heating/cooling of the retentate. All MF was undertaken at 0 Bar (no valve 2, 3 or 5 closure; valve 1 and 4 remained fully closed) and collection of retentate commenced only after permeate composition had equilibrated.

### Microfitration of AMF Serum

The AMF serum was filtrated until a minimal volume of retentate remained, which corresponded to the removal of 35 L permeate. The retentate was diluted with 200 L water and then diafiltrated until a minimal amount of retentate remained.

### Hydrolysis Method

The MF retentate was diluted to approximately 40 L, held at 40° C. and adjusted to approximately pH 8.5. Novozyme PTN Trypsin concentrate (10 g) was dissolved in 100 mL water and then added to the retentate tank. The mixture was incubated for 90 min in the MF plant, which continued to operate throughout to provide mixing. The target conditions were pH 7.5-8.5 and 40° C. Sodium hydroxide (0.29 kg, 2M) was added to maintain the pH. After 90 min the hydrolysate was heated to 52° C. and held for 20 min to deactivate the remaining trypsin.

### Microfitration of Beta-Serum Hydrolysate

The MF retentate hydrolysate was concentrated by the removal of 47 L permeate and then diafiltrated by the addition and removal of 200 L water.

### Drying, Storage and Transport

The material was cooled to 5.7° C. by passing chilled water through the PHE and then freeze-dried for 72 h at 40° C. The dried MF Ret/Hyd/Ret was stored in sealed plastic bags prior to analysis.

### Results & Discussion

The hydrolysis of beta-serum MF retentate with trypsin successfully produced phospholipid enriched material with a high phospholipid content and a high proportion of phospholipid as a percentage of total fat (Table 9.1). This example shows that the proteases suitable for the production of phospholipid enriched material include mammalian proteases and are not limited to proteases of bacterial or fungal origin.

TABLE 9.1
Composition of powders derived from AMF serum by
MF and hydrolysis.

| | | | | AMF | AMF |
|---|---|---|---|---|---|
| | | AMF | AMF | serum | serum |
| | | serum | serum | MF | MF |
| | AMF | MF | MF | RET/HYD/ | RET/HYD/ |
| Component | serum | PERM | RET | PERM | RET |
| Ash (%) | 6.6 | 8.4 | 5.1 | 10.1 | 7.8 |
| Moisture (%) | 1.6 | 2.5 | 1.5 | 2.5 | 1.0 |
| Fat (%) | 14.8 | 0.7 | 30.9 | 1.1 | 60.8 |
| Phospholipids | 11.3 | 0.3 | 22.5 | 0.2 | 52 |
| (%) | | | | | |
| Non-PL fat (%) | 3.5 | 0.4 | 8.4 | 0.9 | 8.8 |
| Cholesterol | 140 | <1 | 310 | 12 | 650 |
| (mg/100 g) | | | | | |
| Lactose (%) | 41 | 80.3 | 4.4 | 7.9 | 0.7 |
| Protein (%) | 30 | 7.6 | 54.9 | 78.3 | 24.7 |
| Phospholipids | 76.4 | 42.9 | 72.8 | 18.2 | 85.5 |
| (% total fat) | | | | | |

### Example 10

### Optimum Filter Porosity for Processing AMF Serum into AMF Serum MF Retentate

In one embodiment of the process for making a phospholipid-enriched product, it may be desired to use two membrane plants fitted with membranes of different porosities. Example 10 aims to determine the optimum membrane porosity for the first membrane filtration step, which creates AMF MF retentate for hydrolysis.

### Method

### Raw Materials

AMF serum (1000 L) was collected from Rochester in a 1000 L container and transported to Food Science Australia (Werribee) by refrigerated road transport.

### Membrane Filtration Methodology

The trial involved four separations of the raw material. A separation was undertaken, the plant (Alcross Pilot MFS-7 (Tetra Pak)) was cleaned and then the filter was changed. The ceramic filters (Membralox) tested had porosities of 0.1 μm, 0.8 μm, 1.4 μm, or 5.0 μm.

### Microfitration of AMF Serum

The AMF serum (200 L) was filtrated until a minimal volume of retentate (approx 35 L) remained. The retentate was diluted with 100 L water and then diafiltrated until a minimal amount of retentate (approx 35 L) remained. Samples of MF permeate and MF retenate were collected.

### Drying, Storage and Transport

The material was frozen and then transported frozen by refrigerated road transport to Cobram. The samples were defrosted by means of mild heat and then transferred to freeze-dryer trays. The samples were freeze dried at 45° C. for 48 h at 1 mBar.

### Results & Discussion

When AMF serum is filtered with a ceramic filter for the purposes of making AMF serum MF retentate, as the first step towards making a phospholipid-enriched product, the optimum filter porosity is 0.1 μm. The 0.1 μm filter increases the phospholipid content of the MF retentate by entrapping all of the phospholipid, while allowing the passage of ash, lactose, protein and a small amount of non-phospholipid fat. EXAMPLES 1, 2, 6, 7, 9, 10 and 11 show that filters with a porosity of 0.1 μm or 0.3 μm are suitable for the MF processing of AMF serum, but EXAMPLE 10 and 11 show that filters with a porosity of 0.8 μm or larger are unsuitable because they allow the passage of phospholipid into the permeate, which both decrease the concentration and yield of phospholipid.

TABLE 10.1
Composition of powders derived from AMF serum by
MF with ceramic filters of different porosities.

| | RETENTATES | | | | PERMEATES | | | |
|---|---|---|---|---|---|---|---|---|
| | FILTER POROSITY (μm) | | | | | | | |
| COMPONENT | 0.1 | 0.8 | 1.4 | 5.0 | 0.1 | 0.8 | 1.4 | 5.0 |
| Ash (%) | 5.5 | 3.4 | 2.2 | 4.6 | 8.3 | 6.8 | 6.7 | 6.6 |
| Moisture (%) | 1.9 | 3.1 | 2.2 | 3.2 | 3.4 | 1.7 | 1.7 | 2.2 |
| Fat (%) | 27.7 | 44.9 | 51.5 | 41 | 0.25 | 14.9 | 16.5 | 17.1 |
| Phospholipids | 18.7 | 6.4 | 5.3 | 7.9 | 0.1 | 11.9 | 10.6 | 11.8 |
| (%) | | | | | | | | |
| Non-PL fat (%) | 9.0 | 38.5 | 46.2 | 33.1 | 0.2 | 3.0 | 5.9 | 5.3 |
| Cholesterol | 290 | 200 | 200 | 210 | 1 | 140 | 140 | 150 |
| (mg/100 g) | | | | | | | | |
| Lactose (%) | 15.4 | 6.1 | 3.6 | 18.1 | 74.8 | 43.2 | 41.6 | 40.6 |
| Protein (%) | 46.0 | 39.3 | 31.6 | 23.3 | 9.9 | 31.7 | 33.1 | 33.2 |
| Phospholipids | 67.5 | 14.3 | 10.3 | 19.3 | 40.0 | 79.9 | 64.2 | 69.0 |
| (% total fat) | | | | | | | | |
| PL (NMR, %) | 14.2 | 4.8 | 4.1 | 5.4 | NA | 8.5 | 9.3 | 9.2 |
| PC (% PL) | 27.5 | 26.4 | 27.7 | 27.7 | NA | 27 | 28 | 27.1 |
| PI (% PL) | 9.2 | 10 | 11 | 9 | NA | 9 | 8.9 | 9.1 |
| PS (% PL) | 12.4 | 12.3 | 13.3 | 11.7 | NA | 11.4 | 11.7 | 12 |
| 2LPC (% PL) | 0.7 | 0 | 0 | 0.9 | NA | 1.1 | 0.6 | 0.6 |
| PE (% PL) | 30.2 | 26.1 | 25.5 | 28.9 | NA | 31.1 | 31.1 | 30.3 |
| SM (% PL) | 16.5 | 19.4 | 18.4 | 17.7 | NA | 16.5 | 16.1 | 17 |
| DHSM (% PL) | 2.9 | 4.1 | 4 | 3.2 | NA | 3.2 | 3 | 3 |
| 2LPE (% PL) | 0.5 | 1.8 | 0 | 0.9 | NA | 0.7 | 0.5 | 0.6 |
| Total SM* | 19.3 | 23.5 | 22.5 | 20.9 | NA | 19.7 | 19.1 | 20 |
| (% PL) | | | | | | | | |

*Total SM = SM + DHSM

### Example 11

### Optimum Filter Porosity for Processing AMF Serum MF Ret/Hyd into PLRDME

### Method

### Raw Materials

AMF serum (1000 L) was collected from Rochester in a 1000 L container and transported to Cobram by refrigerated road transport.

### Primary Microfitration of AMF Serum

AMF serum (800 L) was concentrated to 300 L MF retentate in four 200 L batches by means of a membrane filtration plant. Material in the MF plant was held at between 20° C. and 50° C., whereas material not in the MF plant was refrigerated to ≦4° C.

The membrane filtration plant (Model 92 Laboratory Unit, Filtration Engineering Co. Inc.) was fitted with Desal JX (0.3 μm MF) membranes and a plate heat exchanger (PHE) was fitted to the valve 5 outlet to allow cooling of the retentate. All MF was undertaken at 0 Bar (no valve 2, 3 or 5 closure; valve 1 and 4 remained fully closed) and collection of retentate commenced only after permeate composition had equilibrated.

### Hydrolysis Method

The 300 L MF retentate was transferred to a jacketed and stirred cheese vat, which was subsequently heated to 50° C. and adjusted to pH 9.0 with NaOH (3.3 kg, 2M). Novozyme Alcalase 2.4L FG (500 mL or 0.5 mL Alcalase/mL initial AMF serum) was added directly to the cheese vat. Hydrolysis occurred for 90 min at 50° C. The target was pH 9.0 and NaOH (3.5 kg, 2M) was periodically added to raise the pH, but hydrolysis caused the pH to drop to as low as pH 7.50.

### Pasteurisation and Transport

The MF Ret/Hyd was pumped from the cheese vat into a 1000 L container via a custom manufactured Hipex UHT plant. Passage through the UHT plant pasteurised the MF Ret/Hyd by heating to 72° C. for 18 s and then cooling to 20° C. to reduce product degradation and reduce Alcalase proteolytic activity. The final volume of MF Ret/Hyd was adjusted to 900 L by adding 600 L pasteurised water.

The 1000 L container was transferred to a 2° C. cold room and then transferred to FSA at Werribee by refrigerated road transport.

### Membrane Filtration Methodology

The trial involved four separations of the MF Ret/Hyd. A separation was undertaken, the plant (Alcross Pilot MFS-7 (Tetra Pak)) was cleaned and then the filter was changed. The ceramic filters (Membralox) tested had porosities of 0.1 μm, 0.8 μm, 1.4 μm, or 5.0 μm.

### Microfitration of AMP Serum

The MF Ret/Hyd (200 L) was filtrated until a minimal volume of retentate (approx 35 L) remained. The retentate was diluted with 100 L water and then diafiltrated until a minimal amount of retentate (approx 35 L) remained. Samples of MF Ret/Hyd MF permeate and MF Ret/Hyd MF retenate were collected.

### Drying, Storage and Transport

The material was frozen and then transported frozen by refrigerated road transport to Cobram. The samples were defrosted by means of mild heat and then transferred to freeze-dryer trays. The samples were freeze-dried at 60° C. for 48 h and then 50° C. for 12 h at 1 mBar.

### Results & Discussion

A 0.1 μm filter has the optimum porosity for manufacturing AMF serum MF Ret/Hyd into a phospholipid-enriched product. A 0.1 μm filter gives the highest purity and yield of phospholipids (54 g solids/L at 56% w PL/w solids) by retaining all phospholipids in the retentate, while allowing ash, peptides, protein, lactose and some non-phospholipid fat to move into the permeate. EXAMPLES 1, 2, 4, 6, 7, 9, 10 and 11 show that filters with a porosity of 0.1 μm or 0.3 μm are suitable for the MF processing of AMF serum MF Ret/Hyd, but EXAMPLE 11 shows larger filters with a porosity 0.8 μm or larger are unsuitable because they allow the passage of phospholipid into the permeate, which both decreases the concentration and yield of phospholipid.

TABLE 11.1
Composition of powders derived from AMF serum MF
Ret/Hyd by MF with ceramic filters of different porosities.

| | | AMF | | | | | | | | |
|---|---|---|---|---|---|---|---|---|---|---|
| | | serum | RETENTATES | | | | PERMEATES | | | |
| | AMF | MF RET/ | FILTER POROSITY (μm) | | | | | | | |
| COMPONENT | serum | HYD | 0.1 | 0.8 | 1.4 | 5.0 | 0.1 | 0.8 | 1.4 | 5.0 |
| Ash (%) | 6.8 | 7.7 | 8.1 | 5.5 | 7.3 | 7.2 | 8.9 | 8.2 | 8.1 | 8.0 |
| Moisture (%) | 4.2 | 6.8 | 2.5 | 5.9 | 6.6 | 6.8 | 5.3 | 3.2 | 3.2 | 4.9 |
| Fat (%) | 16 | 23 | 55 | 47 | 32 | 30 | 0 | 23 | 21 | 24 |
| Phospholipids, | 13 | 19 | 56 | 19 | 16 | 16 | <0.1 | 19 | 21 | 20 |
| extraction (%) | | | | | | | | | | |
| Non-PL fat (%) | 2.6 | 3.6 | 0.0 | 28 | 16 | 14 | 0.2 | 3.7 | 0.0 | 4.3 |
| Cholesterol | 140 | 260 | 710 | 280 | 230 | 210 | <1 | 270 | 270 | 280 |
| (mg/100 g) | | | | | | | | | | |
| Lactose (%) | 46 | 21 | 3.2 | 10 | 15 | 19 | 28 | 22 | 22 | 21 |
| Protein (%) | 26 | 36 | 23 | 29 | 33 | 32 | 44 | 38 | 38 | 38 |
| Phospholipids | 83 | 84 | 102 | 41 | 50 | 54 | 0.0 | 84 | 101 | 82 |
| (% total fat) | | | | | | | | | | |
| Phospholipids | 10 | 15 | 49 | 18 | 13 | 12 | NA | 15 | 15 | 16 |
| (NMR, %) | | | | | | | | | | |
| PC (% PL) | 27 | 27 | 27 | 27 | 28 | 27 | NA | 27 | 27 | 27 |
| PI (% PL) | 8.7 | 8.9 | 9.1 | 9.6 | 8.6 | 7.9 | NA | 8.7 | 8.8 | 8.4 |
| PS (% PL) | 12 | 12 | 12 | 13 | 12 | 12 | NA | 12 | 12 | 12 |
| 2LPC (% PL) | 0.6 | 0.8 | 0.6 | 0.8 | 0.6 | 0.7 | NA | 0.9 | 0.7 | 0.7 |
| PE (% PL) | 31 | 28 | 28 | 28 | 28 | 30 | NA | 29 | 29 | 30 |
| SM (% PL) | 17 | 19 | 19 | 17 | 17 | 17 | NA | 18 | 19 | 18 |
| DHSM (% PL) | 3.5 | 3.5 | 3.8 | 3.9 | 3.6 | 3.9 | NA | 3.5 | 3.8 | 3.9 |
| 2LPE (% PL) | 0.5 | 1.0 | 0.8 | 1.1 | 1.3 | 0.8 | NA | 0.9 | 0.8 | 0.6 |
| Total SM* | 20 | 23 | 23 | 21 | 21 | 21 | NA | 22 | 23 | 22 |
| (% PL) | | | | | | | | | | |

| | | | | | | | | |
|---|---|---|---|---|---|---|---|---|
| Yield of solids from | 54 | 17 | 6 | 8 | 30 | 41 | 46 | 51 |
| liquid on drying (g/L) | | | | | | | | |
| Total nitrogen (%) | 3.64 | | | | | | | |
| Non-protein nitrogen (%) | 1.13 | | | | | | | |
| Non-casein nitrogen (%) | 1.17 | | | | | | | |
| Whey protein nitrogen | 0.04 | | | | | | | |
| (%) | | | | | | | | |
| Casein nitrogen (%) | 3.60 | | | | | | | |
| Fatty acid | | | | | | | | |
| 4:0 | 0.8 | | | | | | | |
| 6:0 | 0.8 | | | | | | | |
| 8:0 | 0.5 | | | | | | | |
| 10:0 | 1.2 | | | | | | | |
| 10:1 ω1 cis | <0.1 | | | | | | | |
| 12:0 | 2.1 | | | | | | | |
| 12:1 ω9 cis | <0.1 | | | | | | | |
| 12:1 ω6 cis | <0.1 | | | | | | | |
| 12:1 ω3 cis | <0.1 | | | | | | | |
| 14:0 iso | <0.1 | | | | | | | |
| 14:0 | 7.3 | | | | | | | |
| 14:1 ω5 cis | 0.6 | | | | | | | |
| 15:0 ante-iso | 0.4 | | | | | | | |
| 15:0 | 0.9 | | | | | | | |
| 16:0 | 26.2 | | | | | | | |
| 15:1 ω5 cis | 0.2 | | | | | | | |
| 16:0 iso | <0.1 | | | | | | | |
| 16:1 trans | 0.1 | | | | | | | |
| 16:1 ω7 cis | 1.7 | | | | | | | |
| 17:0 iso | <0.1 | | | | | | | |
| 17:0 ante-iso | <0.1 | | | | | | | |
| 17:0 | 0.6 | | | | | | | |
| 17:1 ω7 cis | 0.3 | | | | | | | |
| 18:0 iso | 0.1 | | | | | | | |
| 18:0 | 11.3 | | | | | | | |
| 18:1 cis | 26.1 | | | | | | | |
| 18:1 trans | 1.0 | | | | | | | |
| 18:2 cis9 tr12 | <0.1 | | | | | | | |
| 18:2 conj | 0.2 | | | | | | | |
| 18:2 tr9 cis12 | <0.1 | | | | | | | |
| 18:2 trans | 0.3 | | | | | | | |
| 18:2 ω6 cis | 5.4 | | | | | | | |
| 18:3 trans | 0.2 | | | | | | | |
| 18:3 ω3 cis | 0.7 | | | | | | | |
| 18:3 ω6 cis | 0.1 | | | | | | | |
| 18:4 ω3 | <0.1 | | | | | | | |
| 20:0 | 0.3 | | | | | | | |
| 20:1 ω9 cis | <0.1 | | | | | | | |
| 20:2 ω6 cis | 0.1 | | | | | | | |
| 20:3 ω3 cis | <0.1 | | | | | | | |
| 20:3 ω6 cis | 0.7 | | | | | | | |
| 20:4 ω3 | <0.1 | | | | | | | |
| 20:4 ω6 cis | 0.6 | | | | | | | |
| 20:5 ω3 cis | 0.2 | | | | | | | |
| 21:5 ω3 | <0.1 | | | | | | | |
| 22:0 | 1.8 | | | | | | | |
| 22:1 ω9 cis | <0.1 | | | | | | | |
| 22:2 ω6 cis | 2.1 | | | | | | | |
| 22:5 ω3 | 0.5 | | | | | | | |
| 22:5 ω6 | <0.1 | | | | | | | |
| 22:6 ω3 cis | <0.1 | | | | | | | |
| 24:0 | 1.4 | | | | | | | |
| 24.1 ω9 cis | 0.2 | | | | | | | |
| Others | 2.9 | | | | | | | |

*Total SM = SM + DHSM

### Example 12

### Optimum Filter Porosity for Processing AMF Serum MF Ret that has been Hydrolyses with Trypsin into PLRDME

### Method

### Raw Materials

AMF serum (1000 L) was collected from Rochester in a 1000 L container and transported to Cobram by refrigerated road transport.

### Primary Microfitration of AMF Serum

AMP serum (1000 L) was concentrated to 220 L, diluted with 370 L diafiltration water and then reconcentrated to 267 L MF retentate in one batch by means of a membrane filtration plant. The membrane filtration plant was a Combi-SW-C1 UF/RO/NF/MF plant (APV Anhydro AS) fitted with three Koch KM membranes (5.8″ spiral, 0.1 μm MF). All MF was undertaken at 2 Bar and 16°-20° C.

### Hydrolysis Method

The 267 L MF retentate was transferred to a jacketed and stirred cheese vat, which was subsequently heated to 45° C. and adjusted to pH 8.7 with NaOH (1.65 kg, 2M). Enzyme Solutions Trypsin 1:250 (0.29 kg or 1% solids [276 L at 10.5 Brix, approximately 29 kg solids]) was dissolved in 5 L water and the enzyme solutions was added directly to the cheese vat. Hydrolysis occurred for 90 min at 45° C. The target range was pH 7.5-8.5 and NaOH (5.5 kg, 2M) was periodically added to raise the pH, but hydrolysis caused the pH to drop to as low as pH 6.9.

### Pasteurisation and Transport

The MF Ret/Hyd was pumped from the cheese vat into a 1000 L container via a custom manufactured Hipex UHT plant. Passage through the UHT plant pasteurised the MF Ret/Hyd by heating to 78° C. for 3 s, then cooling to 44° C. and then further cooling to 6° C. The MF Ret/Hyd was homogenised in two stages while at 44° C. (stage 1, 15 Bar and stage 2, 168 Bar). The final volume of MF Ret/Hyd was adjusted to 900 L by adding 600 L pasteurised water.

The 1000 L container was transferred to a 2° C. cold room and then transferred to FSA at Werribee by refrigerated road transport.

### Membrane Filtration Methodology

The trial involved four separations of the MF Ret/Hyd. A separation was undertaken, the plant (Alcross Pilot MFS-7 (Tetra Pak)) was cleaned and then the filter was changed. The ceramic filters (Membralox) tested had porosities of 0.1 μm, 0.8 μm, 1.4 μm, or 5.0 μm.

### Microfitration of AMF Serum

The AMF serum MF Ret/Hyd (200 L) was filtrated until a minimal volume of retentate (approx 35 L) remained. The retentate was diluted with 100 L water and then diafiltrated until a minimal amount of retentate (approx 35 L) remained. Samples of MF Ret/Hyd MF permeate and MF Ret/Hyd MF retenate were collected.

### Drying, Storage and Transport

The material was frozen and then transported frozen by refrigerated road transport to Cobram. The samples were defrosted by means of mild heat and then transferred to freeze-dryer trays. The samples were freeze-dried at 60° C. for 48 h and then 50° C. for 12 h at 1 mBar.

### Results & Discussion

EXAMPLE 12 confirms the conclusion drawn in EXAMPLE 9 that a phospholipid-enriched product can be prepared if Alcalase is replaced by the mammalian (non-bacterial, non-fungal) protease Trypsin. EXAMPLE 12 also confirms the conclusion from EXAMPLE 11 that 0.1 μm is the optimum porosity for the preparation of a phospholipid-enriched product.

TABLE 12.1
Composition of powders derived from AMF serum MF
Ret when hydrolysed with trypsin and then separated by MF
with ceramic filters of different porosities.

| | RETENTATES | | | | PERMEATES | | | |
|---|---|---|---|---|---|---|---|---|
| | FILTER POROSITY (μm) | | | | | | | |
| COMPONENT | 0.1 | 0.8 | 1.4 | 5.0 | 0.1 | 0.8 | 1.4 | 5.0 |
| Ash (%) | 7.5 | 10 | 14 | NA | 7.2 | 6.8 | 6.8 | 6.9 |
| Moisture (%) | 2.9 | NA | NA | NA | 4.8 | 4.7 | 5 | 4.8 |
| Fat (%) | 64 | 45 | 66 | 28 | 9.8 | 23 | 25 | 29 |
| Phospholipids, | 52 | 18 | 13 | 13 | 4.5 | 22 | 25 | 21 |
| extraction (%) | | | | | | | | |
| Non-PL fat (%) | 11 | 27 | 54 | 14 | 5.3 | 0.6 | 0.1 | 8.3 |
| Cholesterol | 730 | NA | NA | NA | 150 | 350 | 380 | 370 |
| (mg/100 g) | | | | | | | | |
| Lactose (%) | 0.5 | 5.4 | 2.9 | 8.3 | 11 | 8.4 | 8.2 | 8 |
| Protein (%) | 20 | 33 | 17 | 46 | 60 | 51 | 49 | 46 |
| Phospholipids | 83 | 40 | 19 | 49 | 46 | 97 | 99 | 71 |
| (% total fat) | | | | | | | | |

1.-40. (canceled)

41. A method for preparation of a composition comprising at least 30% phospholipid from a dairy product, said method comprising:
contacting the milk product with a protease under appropriate conditions to allow hydrolysis of milk proteins to occur to produce a hydrolysate; and
subjecting the hydrolysate to a filtration step to separate it into a retentate fraction and a permeate fraction, whereby the retentate fraction comprises at least 30% phospholipid and at least some protein is present in the permeate fraction.

42. A method according to claim 41, wherein the protease is trypsin.

43. A method according to claim 41, wherein the protease is from International class EC 3.4.21.62.

44. A method according to claim 41, wherein the milk product is selected from the group consisting of cream, colostrum, milk fat globule membrane (MFGM), AMF serum, whey and whole milk, or processed products thereof.

45. A phospholipid enriched dairy extract obtainable by the method of claim 41.

46. A dairy derived composition comprising at least 40% phospholipid and at least 5% protein, with at least 80% phospholipid as a percentage of total fat in the composition.

47. A composition according to claim 46 comprising at least 40% phospholipid and less than 40% protein.

48. A composition according to claim 46, wherein the ratio of phospholipid to protein is at least 1:1.

49. A composition according to claim 46, wherein the ratio of phospholipid to protein is at least 2:1.

50. A composition according to claim 46, wherein the protein is hydrolyzed.

51. A composition according to claim 50, wherein the protein is hydrolyzed by a protease.

52. A composition according to claim 51, wherein the protease is trypsin.

53. A composition according to claim 51, wherein the protease is from International class EC 3.4.21.62.

54. Use of the extract of claim 45 as a nutraceutical, pharmaceutical, cosmetic ingredient, food, food additive or functional food or as a starting material for the production of liposomes.

55. A nutraceutical, pharmaceutical, cosmetic ingredient, food, food additive or functional food or starting material for production of liposomes comprising the extract of claim 45.

56. A pharmaceutical composition comprising the extract of claim 45 and a pharmaceutically acceptable carrier.

57. A method of treating disorders involving abnormal cellular signalling or cell proliferation, apoptosis, inflammation, and cancer comprising administering an effective amount of the extract of claim 45.

58. A method of promoting memory improvement comprising administering an effective amount of the extract of claim 45.

59. A composition comprising polyunsaturated and saturated phospholipids, which phospholipids are present in the composition in a ratio of saturated phospholipid to monounsaturated to polyunsaturated phospholipid of about 6:3:1 respectively.

60. A composition comprising at least 40% phospholipid and less than 40% protein.
